(19) United States
(12) Patent Application Publication
(10) Pub. No.: US 2016/0145273 A1
(43) Pub. Date: May 26, 2016

(54) PRECATALYST FOR SHIBASAKI'S RARE EARTH METAL BINOLATE CATALYSTS

(71) Applicant: THE TRUSTEES OF THE UNIVERSITY OF PENNSYLVANIA, Philadelphia, PA (US)

(72) Inventors: Patrick Walsh, Bala Cynwyd, PA (US); Eric J. Schelter, Philadelphia, PA (US); Jerome Robinson, Philadelphia, PA (US)

(21) Appl. No.: 14/898,925

(22) PCT Filed: Jun. 23, 2014
(86) PCT No.: PCT/US14/43648

§ 371 Date: Dec. 16, 2015

Related U.S. Application Data
(60) Provisional application No. 61/837,833, filed on Jun. 21, 2013.
(60) Provisional application No. 61/953,085, filed on Mar. 14, 2014.

(51) Int. Cl. C07F 5/00; C07C 67/313; C07B 37/02; C07D 333/22; C07C 45/61; C07B 53/00; B01J 31/22; C07C 239/20

(52) U.S. Cl. CPC C07F 5/003; B01J 31/2213; C07C 67/313; C07C 239/20; C07D 333/22; C07C 45/61; C07B 53/00; C07B 37/02; B01J 2531/38

(57) ABSTRACT

Disclosed herein are schemes for the synthesis of novel hydrogen-bonded rare earth-BINOLate precatalyst complexes, the precatalysts, per se, and their application for the generation of anhydrous REMB catalysts by cation-exchange from metal halides.

Pursuant to 35 U.S.C. §202(c), it is acknowledged that the U.S. Government has rights in the invention described herein, which was made with funds from the National Science Foundation, Grant Nos: CHE-1026553 and CHE-0840428.

### FIELD OF THE INVENTION

This invention relates to the fields of chemistry and asymmetric catalysis. More specifically, the invention provides improved methods for synthesis of asymmetric catalysts and catalysts so produced.

### BACKGROUND OF THE INVENTION

Many therapeutically active compounds are chiral, i.e., they exist as paired enantiomers which are distinguished from one another by the designation R and S, in accordance with the Cahn-Ingold-Prelog notation. Although virtually identical in structure, enantiomers may differ greatly in their pharmaceutical effects. Research over the past several decades has shown that there is a distinct therapeutic advantage to be gained from making an enantiomerically pure, therapeutically active compound.

Multi-functional asymmetric catalysts show marked improvements in reactivity and selectivity over traditional catalysts, due to cooperative activation of reaction partners within a single catalyst framework.¹ Shibasaki's heterobimetallic complexes [M₃(THF)ₙ][(BINOLate)₃RE](REMB; RE=Sc, Y, La—Lu; M=Li, Na, K; B=1,1′-bi-2-naphtholate; RE/M/B=1/3/3; Formula I, below) are the most successful heterobimetallic catalysts, where simple modulation of RE, M, and BINOLate substitution patterns produces a diverse library of catalysts. These privileged frameworks catalyze the formation of C-C and C-E (E=N, O, P, S) bonds with high levels of stereoselection and atom economy.² The products generated by these catalysts have been used as key intermediates toward the synthesis of natural products and biologically active compounds.^{2b, 2e, 2h-k, 3} Despite their exceptional performance, there are several challenges that have prevented the widespread practical application of REMB catalysts.

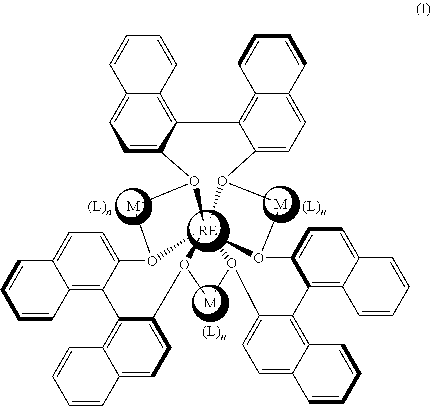

One such challenge arises because both the structure and the catalytic performance of the REMB frameworks are sensitive to trace amounts of moisture.^{2c-e, 2i, 2k, 1, 4} As such, REMB syntheses typically require the rigorous exclusion of water.^{2k-m, 4a, 5} This restriction represents a significant synthetic impediment and also increases the cost of the catalyst, because expensive anhydrous functionalized RE starting materials must be employed rather than inexpensive RE hydrates.^{1d, 6} A key attribute of the REMB catalysts is the tunability in reactivity and selectivity by simply changing RE and M.

Current synthetic strategies to prepare these catalysts, however, require each RE/M combination to be prepared independently. Such an approach is not attractive to high-throughpout experimentation (HTE) strategies,⁷ where ideally a single pre-catalyst could be used to generate multiple catalysts to screen against a large parameter space of reactions and conditions. To overcome these challenges we envisioned air and water-tolerant REMB precatalysts that could provide a rapid simple, user-friendly entry into multiple heterobimetallic frameworks.

While used extensively, synthetic schemes that simplify production of asymmetric catalysts which exhibit high activity, selectivity, and broad substrate generality are highly desirable.

### SUMMARY OF THE INVENTION

The present invention relates to schemes for the synthesis of novel hydrogen-bonded rare earth-BINOLate precatalyst complexes, the precatalysts, per se, and their application for the generation of anhydrous REMB catalysts by cation-exchange from metal halides.

In one aspect, the present invention provides a precatalyst complex of the following formula:

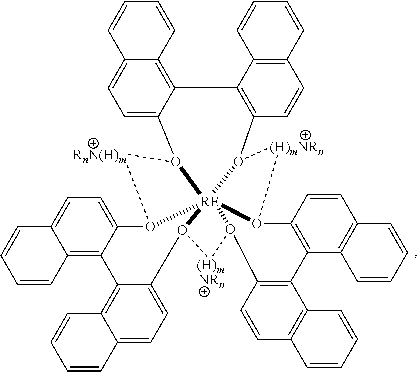

wherein RE represents a rare earth element, NRₙ represents an amine base, m=1 or 2, n=1, 2 or 3 and m+n≦4; and the dashed lines indicate hydrogen bonding which may be monodentate or bidentate hydrogen bonding.

It has been found in accordance with this invention that incorporation of hydrogen-bonded interactions in the secondary coordination sphere of the REMB framework leads to unique properties, most notably, markedly improved stability to the presence of moisture in solution and in the solidstate.

In another aspect, a process for preparing the precatalyst complex is provided. The precatalyst preparation process involves self-assembly of novel hydrogen-bonded rare earth metal BINOLate complexes that serve as bench-stable precatalysts for Shibasaki's REMB catalysts.

Using the precatalysts of this invention, Shibasaki's REMB M=Li⁺, Na⁺, K⁺ frameworks can be quantitatively generated through either acid-base or cation-exchange methods. The approach described herein provides a general strategy to various RE/M combinations without the use of pyrophoric or moisture-sensitive reagents.

### BRIEF DESCRIPTION OF THE DRAWING

FIG. 1 shows numerous chemical syntheses conducted via asymmetric catalysis using REMB catalysts generated from a precatalyst complex of the present invention.

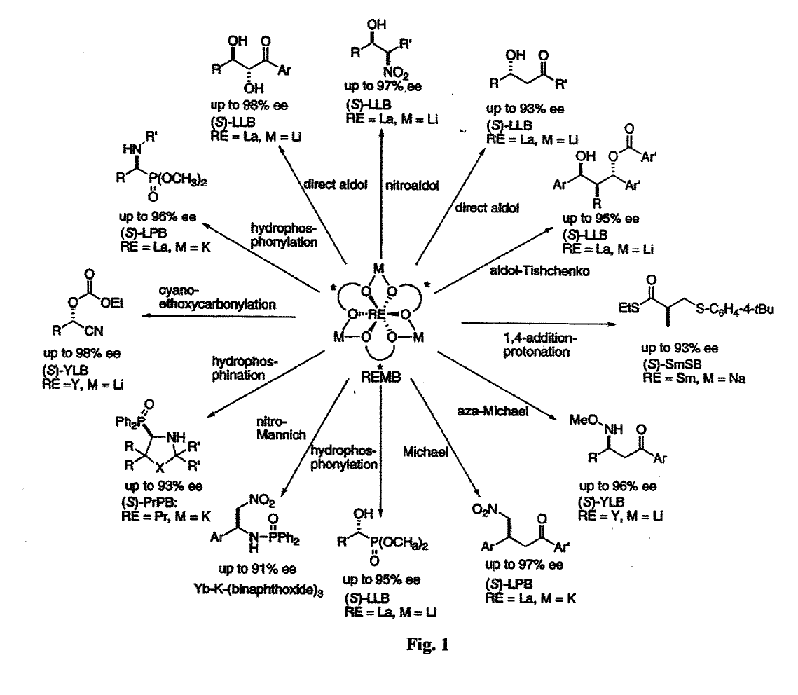

FIG. 2 shows the synthesis of [TMG-H⁺]₃ [RE(BINOLate)₃](1-RE) using rigorously anhydrous conditions.

FIG. 3 (A) is a reaction scheme for generation of 1-RE using hydrated starting materials and conversion to REMB through cation-exchange. (B) Thermal ellipsoid plot (30% probability) of 1-La. (C) ¹H-NMR spectra of 1-Eu (stars) in THF-d₈. (D) ¹H- and ⁷Li{¹H}-NMR (inset) spectra of 1-Eu treated with excess Li in THF-d₈. EuLB (circles) and LiI (square). (E) ¹H- and ⁷Li{¹H}-NMR (inset) spectra in THF-ds of independently synthesized EuLB (circles).

FIG. 4 shows Saa and coworker's RE-BINOLAM framework (RE=Sc, Y, La—Lu; BINOLAM=3,3′-diethylaminomethyl-1,1′-bi-2-naphthol.

### DETAILED DESCRIPTION OF THE INVENTION

Asymmetric catalysis is an attractive method to synthesize optically active materials, which are essential for the production of many pharmaceuticals and fine chemicals. Shibasaki's rare earth-alkali metal-BINOLate framework (REMB; RE=SC, Y and La through Lu; M=Li, Na, K; B=1,1′-Bi-2-naphthol; RE:M:B=1:3:3) is amongst the most successful employed in asymmetric catalysis to date. A library of catalysts are easily generated through simple choice of RE, M, and BINOLate substitution, which has led to the application of these multifunctional catalysts in a wide variety of mechanistically distinct asymmetric reactions from a conserved complex framework. Despite their high level of utility in synthesis, there has not been a simple unified synthetic strategy to provide the anhydrous catalysts without the use of rigorously anhydrous conditions (reagents, solvents, etc.).

The following definitions are used herein:

BAr^{F}=tetrakis-(3,5 trifluoromethyl))borate

BB=Bronstead base

Bn=benzyl

CPME=cyclopentyl methyl ether

DPG=diphenylguanidine

DBU=1,8-diazabicycloundec-7-ene

LA=Lewis acid

OTf=triflate

REMB=Rare earth-alkali metal-BINOLate catalyst framework

sol=solvent

[sub](M)=substrate concentration in moles/liter

THF=tetrahydrofuran

TMG=tetramethylguanidine

Tol=toluene

% ee=percent enantiomeric excess

In accordance with the present invention, schemes are provided for the synthesis of novel hydrogen-bonded rare earth-BINOLate complexes and their application as precatalysts for the generation of anhydrous REMB catalysts by cation-exchange from metal halides and pseudo-halides. Inexpensive rare earth nitrate hydrates and amine bases can be employed to synthesize the precatalyst in high yields using operationally simple and rapid procedures. Among the amine bases that have been used in preparing the precatalyst complexes described herein are guanidines, amidines (both cyclic and non-cyclic) and heterocyclic amines. Representative examples include 1,1′,3,3′-tetramethylguanidine (TMG), and 1,8-Diazabicycloundec-7-ene (DBU), diphenylguanidine (DPG), pyrrolidine and piperidine.

Furthermore, the complex can be isolated from acetonitrile as a precipitate instantaneously and is readily recrystallized as an anhydrous material. Solvents containing water can be used for the same process with no reduction in yield or purity. This approach effectively increases the utility of the catalysts by lowering the economic and equipment barriers for their use. The precatalysts can be employed in mechanistically different reactions with various RE, M, and BINOLate substitution. The precatalyst system offers a unified approach to access different RE/M combinations from a single RE precatalyst source. Results from preliminary studies show negligible to minimal losses in selectivity, validating the efficacy of these complexes as precatalysts for the well-established REMB system.

Positions 5-8 of the (S)BINOL moiety of structural formula I may be substituted with one or more suitable substituent groups, including halogens, e.g., chlorine or bromine, alkyl (C₁-_{C4}) or alkoxy.

The precatalyst of the present invention can be used to generate catalysts which are effective in a number of commercially important chemical syntheses involving asymmetric catalysis. These include organic name reactions, such as the Michel addition reaction and the Diels-Alder reaction.

The Michel addition reaction involves base-promoted conjugate addition of carbon nucleophiles, also referred to as donors, to activated, unsaturated compounds, also referred to as acceptors. Representative donors include malonates, cyanoacetates, acetoacetates, carboxylic esters, ketones, aldehydes, nitriles, nitro compounds and sulfones, to name a few.

Representative acceptors include α,β-unsaturated ketones, esters, aldehydes, amides, carboxylic acids, sulfoxides, sulfones, nitro compounds, phosphonates and phosphoranes, to name a few.

Suitable bases include NaOCH₂CH₃, NH(CH₂CH₃)₂, KOH, KOC(CH₃)₃, N(CH₂CH₃)₃, NaI, Nah, BuLi and lithium diisopropylamide (LDA). See Michael, J. Prakt. Chem. [2]35: 349 (1887).

The Diels-Alder reaction involves the 1,4-addition of the double bond of dienophile to a conjugated diene to yield a 6-membered ring compound, such that up to four new stereo centers may be created simultaneously. The [4+2]-cyclo addition usually occurs with high region and stereoselectivity.

See Diels and Alder, Ann., 460: 98 (1928); 470: 62 (1929); and Ber., 62: 2081, 2087 (1929).

The precatalysts described herein also perform with comparable or improved levels of selectivity in aza-Michael additional reactions and direct Aldol reactions.

In experiments conducted to date, it has been found that installation of hydrogen bond donors enable greater structural control of the rare earth BINOLate complexes of the invention. The present inventors have recently reported^{5c, 8} the results of experiments demonstrating the importance of non-covalent interactions in the secondary coordination sphere with respect to tuning the reactivity and properties of REMB frameworks. In these examples, the alkali metal cations modulate the electronics at the RE cation and BINOLate oxygen atoms, and are the primary determinant for the ability of the RE cation to act as a Lewis acid. Given these observations, we hypothesized that the isoelectronic replacement of alkali metal cations with the appropriate choice of ammonium cations would result in the formation of complexes with ionic H-bonding networks.⁹ Hydrogen-bonds (H-bonds) are essential non-covalent interactions that can direct self-assembly processes and stabilize reactive fragments in Nature and synthetic systems.^{9b, 10} The strength of H-bonding varies greatly with directionality and charge of the donor/acceptor pair, where bond strengths of up to ˜35 kcal/mol can be found for ionic/charged systems.⁹ We expected these relatively weak interactions should allow for facile exchange of H-bonded ammonium cations for alkali metal cations, which would provide a rapid and unified entry to various REMB frameworks. With this approach in mind, we embarked on the synthesis of REMB precatalysts supported by hydrogen-bonds.

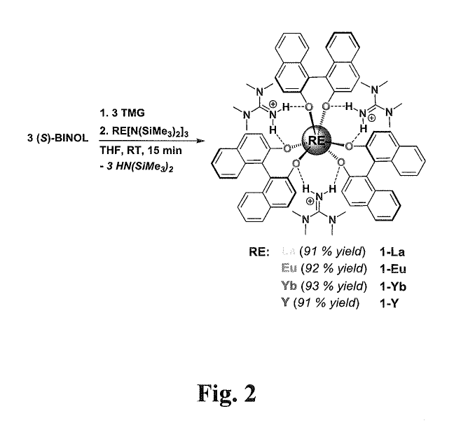

Commercially available 1,1,3,3-tetramethylguanidine (TMG) appeared as an ideal candidate for our synthetic investigation, because when protonated it is a dual H-bond donor that could replace the interactions of the main group metal with two BINOLate ligands (see Formula I, above) in REMB complexes. TMG is sufficiently basic, with a pKₐ(TMG-H⁺)=13.6 in H₂O,¹¹ to deprotonate the phenolic BINOLate hydrogens, given their pKₐ(ArOH)=10.0 in H₂O.¹² Guanidines are known H-bond donors for a variety of anionic hosts.^{10c, 13} Under anhydrous conditions, addition of three equiv TMG to a mixture of one equiv RE[N(SiMe₃)₂]₃ and three equiv (S)-BINOL in THF resulted in instantaneous and quantitative formation of a new 1:3:3 complex, [TMG-H⁺]₃[RE(BINOLate)₃](1-RE), RE=La, Eu, Yb, Y. Removal of the volatiles followed by dissolution of the residue in CH₂Cl₂ and layering with pentane furnished 1-RE in excellent crystalline yields: 1-RE; RE=La, 91%, Eu, 92%, Yb, 93%, Y, 91% (FIG. 2).

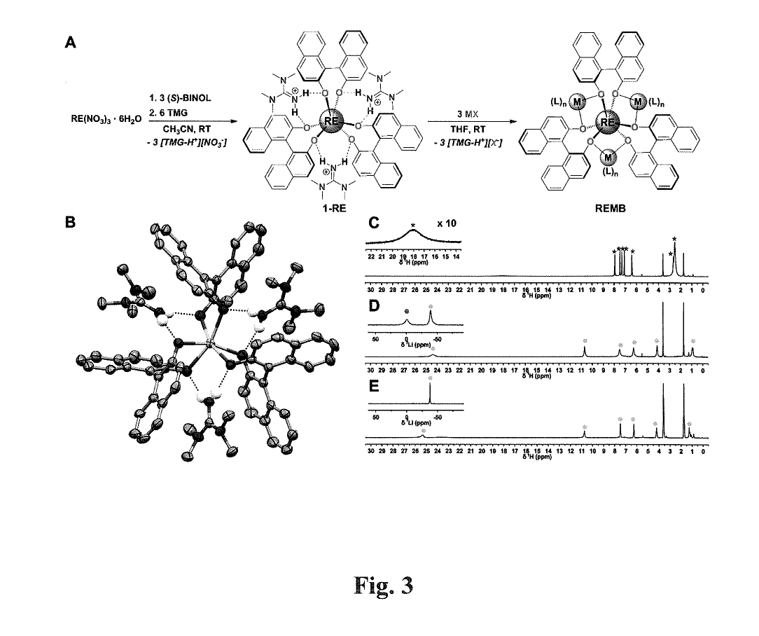

Single crystal X-ray diffraction data for 1-La supported the formation of a 1:3:3 complex (FIG. 3b). The primary coordination sphere at the La(III) cation formed a distorted octahedron consisting of the six-BINOLate oxygen atoms. RE-O_{BINOLate} distances ranged from 2.3996(15)-2.4154(14) Å, similar to reported six-coordinate REMB frameworks^{4a, 5, 8a, 14} after accounting for differences in ionic radii of the RE cations.¹⁵ As expected, the tetramethylguanidinium cations were engaged in bifurcated H-bonding interactions, where each guanidinium cation participated in two H-bonds with neighboring anionic BINOLate oxygen atoms. The N_{TMG}—H . . . O_{BINOLate} distances ranged from 2.782(2) to 2.811(2), and were consistent with reported charged guanidinium N⁺—H . . . O⁻ hydrogen bonds.^{13a, 16}

¹H and ¹³C{¹H}-NMR spectra were consistent with D₃ symmetric 1-RE complexes in solution. The ¹H-NMR spectra revealed six sharp BINOLate resonances and two resonances belonging to the methyl and ammonium protons of TMG-H⁺ (FIG. 3c). Given the importance of Lewis base coordination at the central RE, binding studies were pursued with the paramagnetic analogues, 1-Eu and 1-Yb. Contrary to RE/Li frameworks, addition of cyclohexenone to 1-Eu and 1-Yb resulted in negligible shifts (≦0.012 ppm) of the alkenyl protons (data not shown), which suggested that no binding of the cyclohexenone occurred at the RE center.

In view of the inability of 1-RE to bind cyclohexenone, we extended our investigations to a smaller Lewis base, H₂O. While H₂O can coordinate to REMB systems,^{2c, 4a} partial ligand hydrolysis occurs where the formation of polynuclear hydroxide clusters have been observed and characterized in the solid state.^{4b} Addition of H₂O (0-200 equiv) to 1-RE does not result in the appearance of free protonated BINOL in the ¹H-NMR, nor does it induce formation of multi-RE cation cluster compounds as observed with the REMB frameworks.

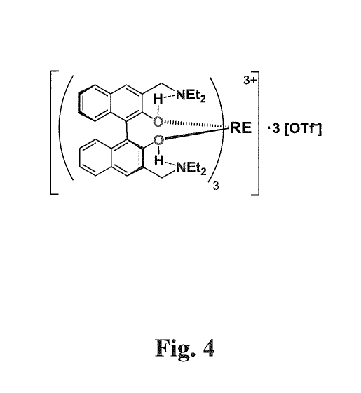

The water tolerance of 1-RE is exceptional, especially when considering the disparate behavior observed for Saa and coworker's RE-BINOLAM system (BINOLAM=3,3′-diethylaminomethyl-1,1′-bi-2-naphthol; RE:BINOLAM=1:3, FIG. 4).¹⁷ In contrast to 1-RE, RE-BINOLAM contains neutral intramolecular H-bonding pairs that consist of phenolic OH donors and alkyl amine acceptors. The RE-BINOLAM complexes are highly sensitive to ligand hydrolysis; synthesis of RE-BINOLAM complexes require rigorous exclusion of water, while the generation of free ligand from a hydrolysis event can be observed even in dry CD₃CN.^{17c}

The water tolerance of 1-RE is attributed by the present inventors to the strong preference for a six-coordinate geometry at the RE cation. Both RE-BINOLAM and REMB complexes will coordinate H₂O to adopt seven-coordinate geometries.^{2c,4a,17c} The acidity of H₂O coordinated to RE cations is increased by ˜5-6 orders of magnitude,¹⁸ resulting in enhanced rates of ligand hydrolysis. We propose that the coordination preferences in 1-RE arise from the unique intramolecular, ionic H-bonding interactions. The H-bond donors, H-TMG⁺, assume geometries in the solid state that maximize the strength of the directional H-bonding interactions. Coordination of H₂O or other Lewis bases at the RE³⁺ cations would increase the energy of the system by weaking those intramolecular H-bonding interactions, disfavoring the seven-coordinate geometries for 1-RE.

Encouraged by the moisture stability of 1-RE, the present inventors pursued a modified, open-air, benchtop synthesis using inexpensive hydrated RE starting materials. By taking advantage of the rapid kinetics associated with complex formation and the low solubility of 1-RE in polar solvents, a convenient and expedient synthetic procedure was identified. Addition of six equiv TMG to concentrated stirring solutions of RE(NO₃)₃.6H₂O/(S)-BINOL (1:3 ratio) resulted in the immediate precipitation 1-RE, which could be crystallized from CH₂Cl₂/pentane in 70-85% yield. Using these conditions 1-La was easily prepared on a 25 g scale (FIG. 3a). Other early REs (La—Eu) were accessible following this procedure, with 1-Eu reported as a representative, fully characterized example obtained in 79% crystalline yield.

The successful synthesis of 1-RE from hydrated starting materials was surprising, because of the high hydration enthalpies associated with RE³⁺ cations^{18a,48} and the aqueous speciation of RE(NO₃)₃, that tend to form RE(NO₃)ₓ(OH)_{y-x} compounds at neutral or basic pH following acid hydrolysis.¹⁹ In this context, the increased Lewis acidity of the late lanthanides (Gd—Lu) and Y proved problematic for their open-air syntheses of 1-RE, where unlike the early lanthanides, the formation of an inseparable byproduct (˜30%) was observed. Suppression of this byproduct, likely a mixed hydroxide species, was possible by lowering the pH of the RE(NO₃)₃.XH₂O solution with three equiv acetic acid. Addition of a CH₃CN solution of three equiv (S)-BINOL and six equiv TMG to the acidified RE(NO₃)₃.XH₂O solution, followed by neutralization with an additional three equiv TMG resulted in the rapid formation and precipitation of 1-RE. Crystallization from CH₂Cl₂/pentane furnished 1-RE in similarly high yields, 1-RE: Y=85%, Yb=80%, where 1-Y was synthesized on a 10 g scale.

Notably, the synthesis of 1-RE from either method could be carried out using technical-grade solvents without additional drying, and provided anhydrous, crystalline products following mild drying conditions (˜50° C., 200 mTorr, 2 h). Unlike the REMB or RE-BINOLAM complexes, no coordinated or interstitial H₂O crystallized with 1-RE synthesized from benchtop methods.^{2c,4a,17c} In addition to the strong preference for a six-coordinate geometry of the RE cation in 1-RE, we propose that the hydrophobic methyl substituents of TMG-H⁺ contribute to the nonhygroscopic properties of 1-RE

The following examples describe the invention in further detail. These examples are provided for illustrative purposes only, and should in no way be construed as limiting the scope of the invention.

### Example 1

### Generation of REMB from 1-RE

After establishing a practical synthetic protocol for the generation of 1-RE, we investigated methods to access Shibasaki's heterobimetallic catalysts using 1-RE as starting materials. While the ionic H-bonding interactions in 1-RE appeared to confer stability against hydrolysis, we envisioned that the large enthalpic contribution from forming new M-O_{BINOLate} bonds should provide a strong thermodynamic driving force for the formation of the REMB complexes. Indeed, installation of M⁺ was possible through either acid-base or cation-exchange methods, which produced REMB along with three equiv TMG or [TMG-H⁺][X⁻](FIG. 3a). A representative example is shown in FIG. 3c-e, where addition of excess LiI to 1-Eu immediately generates EuLB as the single observable Eu-containing product. Notably, the presence of coordinated water to the REMB was not observed by ¹H NMR using 1-RE synthesized from rigorously anhydrous or benchtop methods, supporting the anhydrous and nonhygroscopic nature of 1-RE (FIG. 3C-E).

Synthetic details and characterizations of the resulting products are set forth below, under the heading Experimental Procedures.

While syntheses of RE heterobimetallic frameworks have been achieved through acid-base, redox, ligand-exchange, or metathetical synthetic routes,²⁰ to the best of our knowledge, there have been no reports using cation-exchange from a RE/ammonium precursor. Our method provides a new and complementary approach that offers several potential advantages compared to traditional synthetic strategies. A large variety of inexpensive MX salts and amine bases of varying pKₐ are commercially available, which should expedite the identification of new heterobimetallic frameworks. Moreover, operational simplicity is also greatly improved by avoiding the use of strong bases that are typically moisture sensitive.

### Example 2

### Catalytic Investigations of 1-La/MX Precatalyst System

Given the rapid and clean conversion of 1-RE to various REMB products through cation metathesis, we turned our attention to identifying conditions where 1-RE could be used as a general precatalyst for REMB reactivity. As an initial trial, the asymmetric Michael-addition was chosen due to its synthetic utility,^{1c, 21} and the sensitivity of the Lewis-acid/Brønsted-base mechanism to catalyst structure, especially in REMB frameworks.^{2c} While we demonstrated REMB can be generated from 1-RE and MX, the optimal combination of MX source, solvent, and additives necessary to ensure the best catalytic performance was unclear at the onset. Given the large number of available combinations of RE and the main group metal, we employed microscale high-throughput experimentation (HTE) techniques⁷ to identify conditions for 1-La/NaX as a precatalyst for LaNaB. A variety of NaX sources were screened with THF or toluene as solvent using cyclohexenone (2a) and dibenzylmalonate (3c) as model substrates. The optimization results for this study are presented in Table 1.

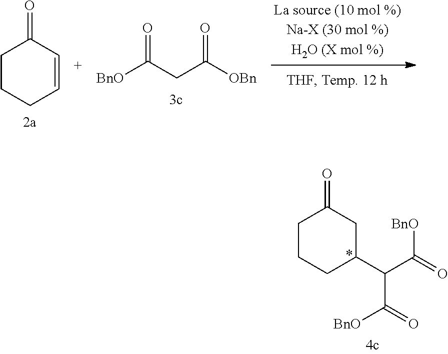 [SMILES: O=C1C=CCCC1 ; O=C(CC(=O)OCc1ccccc1)OCc1ccccc1 ; O=C1CCCC(C(C(=O)OCc2ccccc2)C(=O)OCc2ccccc2)C1]

TABLE 1
Optimization of 1-La/MX in the asymmetric Michael addition.

| | La | | H₂O | Temp | |
|---|---|---|---|---|---|
| Entry | Source | Na—X | (X mol %) | (° C.) | ee (%) |
| 1 | 1-La | — | 0 | 25 | 14 |
| 2 | 1-La | Cl | 0 | 25 | 8 |
| 3 | 1-La | I | 0 | 25 | 42 |
| 4 | 1-La | BAr₄^{a} | 0 | 25 | 33 |
| 5 | 1-La | N(SiMe₃)₂ | 0 | 25 | 50 |
| 6 | LaNaB | — | 0 | 25 | 62 |
| 7 | 1-La | I | 10 | 25 | 70 |
| 8 | 1-La | I^{b} | 10 | 25 | 69 |
| 9 | 1-La | I | 20 | 25 | 75 |
| 10 | 1-La | I | 30 | 25 | 78 |
| 11^{c} | 1-La | I | 30 | 0 | 88 |
| 12^{c} | LaNaB | — | 30 | 0 | 88 |

^{a}Ar = 3,5-(CF₃)₂—C₆H₃
^{b}60 mol % Nal was used instead of 30 mol %
^{c}Malonate added portion wise over 20 min.

As a control experiment, we screened the hydrogen-bound complex 1-La in asymmetric Michael addition. 1-La was not a competent catalyst for the formation of Michael adduct 4c (entry 1). As revealed in our binding studies, 1-La is sterically saturated and would not be expected to provide dual activation of the electrophile and nucleophile required for the Lewis-acid/Brønsted-base mediated mechanism. Inorganic salts such as NaCl (entry 2) were ineffective in generating an active catalyst, however, use of more soluble salts provided 4c in moderate levels of enantiomeric excess (entries 3-5). Using independently prepared LaNaB, we discovered that rigorously anhydrous conditions resulted in only moderate ee's, suggesting the reactions conducted in the original reports on the activity of LaNaB contained trace amounts of water (entry 6). Use of varying amounts of water as an additive (entries 7, 9, 10; additional data not shown) resulted in the identification of an optimal [La]:[H₂O] ratio of 1:3. Addition of excess NaI did not negatively impact selectivity (entry 8), however, selectivities were lower than the original report.^{2c} Ultimately, we found that slow addition of malonate was critical to obtain high levels of enantioselectivity, a key observation which was made by Shibasaki and coworkers for more reactive Michael partners.²² At 0° C., the use of 10 mol % 1-La/NaI or LaNaB (entries 11 and 12) provided identical ee's as observed in the original report of LaNaB.

The generality of the 1-La/NaI precatalyst system was investigated by exploring the scope of Michael donors (Table 2). While the performance of the precatalyst system, 1-La/NaI, matched LaNaB in the Michael addition of 3c to 2a (entry 5), examination of other symmetrical malonates (3a, 3b, 3d) resulted in improved levels of stereoselectivity (94-96% ee entries 1, 4, 6) compared to literature reports (Table 2, values in parenthesis).^{2c}We proposed that the increased selectivity is due to the high purity of LaNaB generated from the 1-La/NaI system (data not shown).

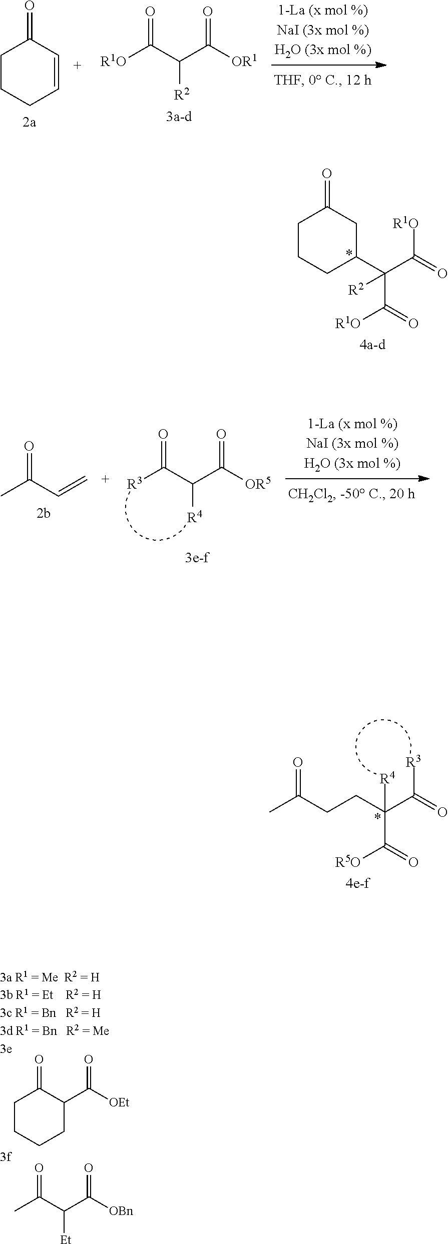

TABLE 2
Asymmetric Michael addition of 1,3-dicarbonyls
to enones with the 1-La/NaI precatalyst system.

| | Michael | | | Yield | ee |
|---|---|---|---|---|---|
| Entry | Donor | Product | x | %^{a} | %^{a} |
| 1 | | | 10^{b} | 87 | 94 |
| | | | | (98)^{c} | (83)^{c} |
| 2 | 3a | 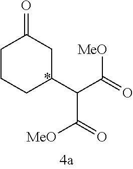 [SMILES: COC(=O)C(C(=O)OC)C1CCCC(=O)C1] | 5^{d} | 93 | 90 |
| 3 | | | 2.5^{d} | 91^{e} | 89^{e} |
| 4 | 3b | 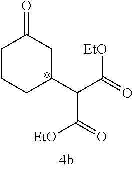 [SMILES: CCOC(=O)C(C(=O)OCC)C1CCCC(=O)C1] | 10^{b} | 90 (97)^{c} | 96 (81)^{c} |
| 5 | 3c | 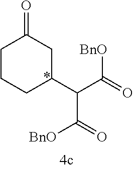 [SMILES: O=C1CCCC(C(C(=O)OCc2ccccc2)C(=O)OCc2ccccc2)C1] | 10^{b} | 94 (97) | 88 (88) |
| 6 | 3d | 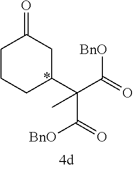 [SMILES: CC(C(=O)OCc1ccccc1)(C(=O)OCc1ccccc1)C1CCCC(=O)C1] | 10^{b} | 89 (91) | 96 (92) |
| 7 | 3e | 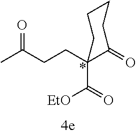 [SMILES: CCOC(=O)C1(CCC(C)=O)CCCCC1=O] | 5^{d} | 87 (89) | 98 (91) |
| 8 | 3f | 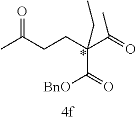 [SMILES: CCC(CCC(C)=O)(C(C)=O)C(=O)OCc1ccccc1] | 5^{d} | 84 (98) | >99 (89) |

^{a}Reactions conducted on a 1.0 mmol scale using 1 equiv of 2a and 2 equiv of 3a-d, or 1.2 equiv of 2b and 1 equiv of 3e, f unless otherwise specified. Values in parenthesis are from ref. 2c and 22 using LaNaB as a catazlyst. Isolated yield after chromatographic purification.
^{b}Michael Donor added portionwise over ~20 minutes.
^{c}Reported reaction ran at room temperature.
^{d}Michael Donor added slowly over 8 h.
^{e}Ran on 1 g scale. A single recrystallization furnished prouct in 87% yield with 94% ee.

In light of the excellent levels of activity and selectivity for 1-La in the optimized Michael addition, we sought to improve the practicality of the system by reducing catalyst loading and examining the scalability of this reaction to produce 4a (Table 2, entries 1-3). Compound 4a has been used as a key intermediate in the enantioselective syntheses of diverse products²³ including strychnine alkaloids,^{3d, 24} (−)-Gilbertine,²⁵ Haouamine B,²⁶ and (+)-2-deoxyolivin.²⁷ Decreased catalyst loadings were possible from the 1-La precatalyst (entries 2 and 3), where 2.5 mol % loading furnished 4a on an 8.7 mmol scale under highly-concentrated reaction conditions²⁸ with minimal losses in enantioselectivity (entry 3). The original levels of selectivity could be restored by a single recrystallization of 4a in 87% yield and 94% ee. While LaNaB is not as effective for this particular transformation as Shibasaki's ALB catalyst, [Li(THF)₃][(BINOLate)₂Al],²⁹ the 1-La/NaI system is an operationally simple complement, because no pyrophoric materials are necessary for the catalyst synthesis.

While a number of highly enantioselective catalysts for the asymmetric Michael addition of malonates to cyclic enones have been identified,^{21a-c, 30} the corresponding addition of β-ketoesters to acyclic enones still remains challenging.³¹ Shibasaki and coworkers reported high levels of stereoselectivity for the addition of β-ketoesters to acyclic enones in CH₂Cl₂ with as little as 5 mol % LaNaB as a catalyst.²² Employing the 1-La/NaI precatalyst system under similar conditions, addition of cyclic (3e) and acyclic (3f) Michael donors to methyl vinyl ketone (2b) furnished Michael adducts 4e and 4f in 98 and 99% ee respectively (entries 7 and 8). A similar ˜10% improvement in ee was observed from the original report,²² suggesting that this phenomena could be observed in other Lewis-acid/Brønsted-base reactions.

A key attribute of the REMB system is the diversity observed in the catalytic reactions upon changing RE and M combinations. To establish that our precatalyst is amenable to different RE/M combinations, we investigated the Lewis-acid/Lewis-acid mediated aza-Michael addition of O-methylhydroxylamine to α,β-unsaturated ketones.^{2h, 26} Shibasaki and coworkers accessed optically active β-amino carbonyl compounds using low catalyst loadings of YLB (0.5-3.0 mol %). In addition, β-amino carbonyl compounds are important structural motifs in many biologically active compounds.³² Shibasaki and co-workers demonstrated that their products could be further transformed to other useful chiral building blocks such as aziridines or β-amino alcohols with no loss in ee.^{2k, 33}

Application of the optimized 1-RE/MI precatalyst system to generate YLB from 1-Y/LiI proved general. At 3 mol % loading of 1-Y, comparable selectivities were obtained for various substitution patterns (Table 3, 7a-e) including examples of an electron-donating group (7b), electron-withdrawing group (7c), heterocycle (7d), and extended conjugation (7e). The scalability of this reaction was also maintained, where 7a could be obtained in 93% yield and 93% ee on a 1 g scale (entry 2). Catalyst loading could be further reduced to 0.5 mol % (entry 3), albeit with slightly decreased levels of enantioselectivity (88% versus 93% ee).

Additives have played an important, and at times poorly understood, role in improving the performance of the REMB catalysts.^{1a, 2i, 21-n, 34} For example, the addition of MOH and H₂O to REMB solutions can generate highly active second generation catalysts for aldol and nitroaldol reactions.^{1a, 2i} To test the compatibility of additives in our precatalyst system, we chose the direct aldol reaction catalyzed by second generation LaLB (LaLB.KOH). Addition of 8 mol % KO^{t}Bu and 16 mol % H₂O to 1-La/LiI (8/24 mol %) generated LaLB.KOH, which catalyzed the direct aldol reaction between pivaldehyde (8) and acetophenone (9) to furnish 3-hydroxy-4,4-dimethyl-1-phenylpentan-1-one (10) in 74% yield and 95% ee (Scheme 1). Interestingly, our preliminary results revealed an improvement (˜7% ee) in

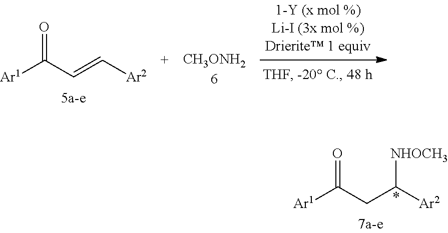

TABLE 3
Asymmetric aza-Michael Addition of O-Methylhydroxylamine to
Chalcone Derivatives with the 1-Y/LiI Precatalyst System

| | | | Yield | ee |
|---|---|---|---|---|
| Entry | Product | x | %^{a} | %^{a} |
| 1 | | 3 | 88 | 91 |
| | | | (97) | (95) |
| 2 | 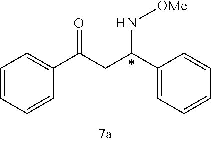 [SMILES: CONC(CC(=O)c1ccccc1)c1ccccc1] | 3 | 93^{b} | 93^{b} |
| 3 | | 0.5 | 90^{b,c} | 88^{b,c} |
| | | | (96) | (96) |
| 4 | 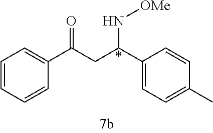 [SMILES: CONC(CC(=O)c1ccccc1)c1ccc(C)cc1] | 3 | 91 (96) | 94 (96) |
| 5 | 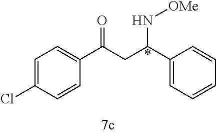 [SMILES: CONC(CC(=O)c1ccc(Cl)cc1)c1ccccc1] | 3 | 96 (96) | 94 (96) |
| 6 | 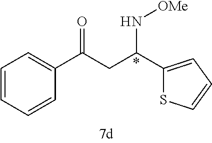 [SMILES: CONC(CC(=O)c1ccccc1)c1cccs1] | 3 | 93 (96) | 93 (95) |
| 7 | 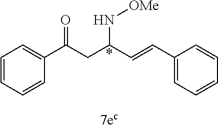 [SMILES: CONC(/C=C/c1ccccc1)CC(=O)c1ccccc1] | 3 | 97^{d} (91) | 91^{d} (94) |

^{a}Reactions conducted on a 0.5 mmol scale using 1 equiv of 7a-e and 1.2 equiv of 8 in THF ([enone] = 1.6M) unless otherwise specificed. Values in parentheses are from using YLB (refs 2k and 34). Isolated yield after chromatographic purification.
^{b}1 g scale.
^{c}80 h, [enone] = 2.05M.
^{d}60 h, [enone] = 1M.

enantioselectivity using our precatalyst system in a second Lewis-acid/Brønsted-base catalyzed reaction, which supports that our system is amenable to additives similar to those of the REMB framework.

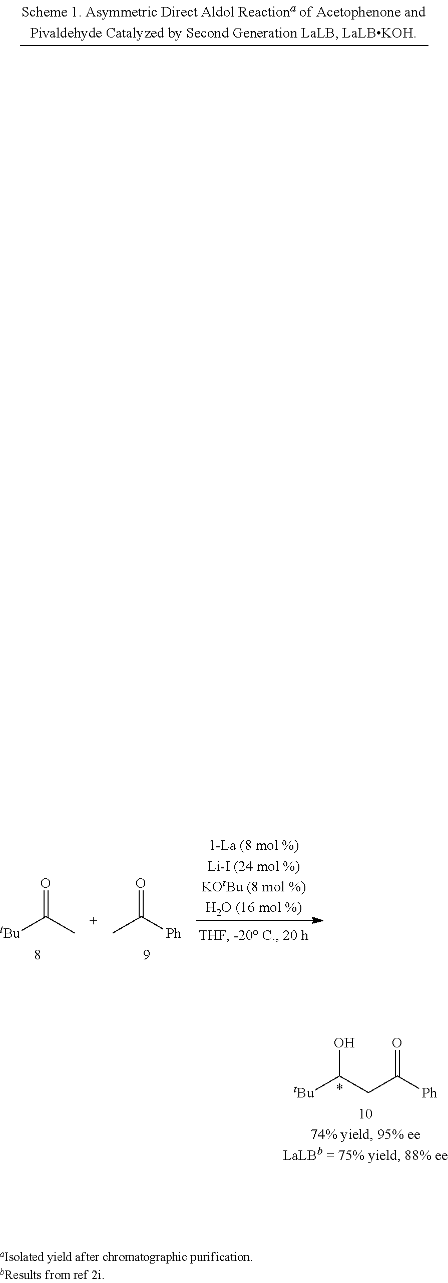

While the REMB catalysts can be stored at room temperature for extended periods of time under a dry N₂ atmosphere with no significant loss in catalytic activity, we were interested in performing a side-by-side comparison of the stability of 1-RE and REMB as solids stored on the bench-top. Crystals of 1-RE and REMB were stored in vials exposed to open air for six months and then employed in each of the mechanistically distinct reactions (Scheme 2). 1-RE/MX precatalysts maintained excellent catalytic activity, whereas the performance of REMB were significantly reduced due to the decomposition associated with prolonged exposure to ambient atmosphere.

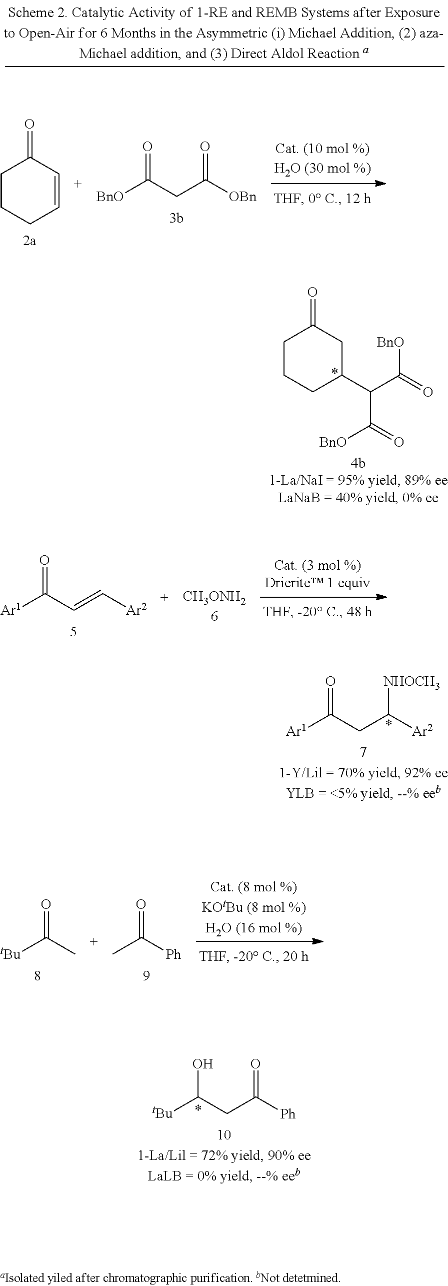

The foregoing examples further support the tolerance of 1-RE to bench-top conditions, and highlight their suitability as robust precatalysts.

### Experimental Procedures

General Methods.

For all reactions and manipulations performed under an inert atmosphere (N₂), standard Schlenk techniques or a Vacuum Atmospheres, Inc. Nexus II drybox equipped with a molecular sieves 13×/Q5 Cu-0226S catalyst purifier system were used. Glassware was oven-dried overnight at 150° C. prior to use. ¹H- and ¹³C{¹H}NMR spectra were obtained on a Brüker AM-500 or Brüker UNI-400 Fourier transform NMR spectrometer at 500 and 126 MHz or 400 and 101 MHz, respectively. ⁷Li{¹H}-NMR were recorded on a Brüker AM-500 or Brüker UNI-400 Fourier transform NMR spectrometer at 194 MHz and 155 MHz respectively. All spectra were measured at 300 K unless otherwise specified. Chemical shifts were recorded in units of parts per million downfield from residual proteo solvent peaks (¹H—) or characteristic solvent peaks (¹³C{(¹H}). The ⁷Li{¹H} spectra were referenced to external solution standards of LiCl in H₂O (at zero ppm). All coupling constants are reported in hertz. The infrared spectra were obtained from 400-4000 cm⁻¹ using a Perkin Elmer 1600 series infrared spectrometer. Elemental analyses were performed at the University of California, Berkeley Microanalytical Facility using a Perkin-Elmer Series II 2400 CHNS analyzer. High-resolution mass spectra were measured using a Waters 2695 Separations Module (1S0RR23444). All high-throughput experiments (HTE) were set up inside a Vacuum Atmospheres glovebox under a nitrogen atmosphere. The experimental design was accomplished using Accelrys Library Studio. Liquids were dispensed using multi-channel or single-channel pipettors. Solid chemicals were dosed as solutions or slurries in appropriate solvents.

Compounds 4a-f,^{2c,22} 7a-e,^{2k} and 10²ⁱ have been previously reported. Absolute configurations of Compounds 4a-e,^{2c,36} 7a-b^{2k} have previously been determined. The absolute configuration of Compound 4f was tentatively assigned on the basis of the previous results,²² which used the opposite enantiomer, (RRR)—LaNaB, from that of our studies. Absolution configurations of Compounds 7c-f were not previously assigned,^{2k} but used the same enantiomer, (SSS)-YLB, as our report. The absolute configuration of Compound 10 was not previously assigned,²ⁱ but used the opposite enantiomer, (RRR)—LaLB, from that of our studies.

Materials.

Tetrahydrofuran, diethyl ether, dichloromethane, hexanes, and pentane were purchased from Fisher Scientific. The solvents were sparged for 20 min with dry N₂ and dried using a commercial two-column solvent purification system comprising columns packed with Q5 reactant and neutral alumina respectively (for hexanes and pentane), or two columns of neutral alumina (for THF, Et₂O and CH₂Cl₂). Solvents (CH₂Cl₂, CH₃CN, and pentane; ACS grade, FisherSci) and 1,1,3,3-tetramethylguanidine (Acros) were purchased and used in General Procedure B without further purification. Deuterated tetrahydrofuran and chloroform were purchased from Cambridge Isotope Laboratories, Inc. and stored for at least 12 h over potassium mirror or 4 Å molecular sieves, respectively, prior to use. 1,1,3,3-Tetramethyl guanidine used in General Procedure A was purchased from Acros and degassed using three freeze-pump-thaw cycles and stored for 24 h over 4 Å molecular sieves. (S)-BINOL and RE(NO₃)₃.6H₂O (>99.9% purity; RE=La, Eu, Yb, Y) were purchased from AKScientific and Strem, respectively, and used without additional purification. LiI, NaI, and KO^{t}Bu were purchased from Acros and used without additional purification.

Cyclohexenone (2a), methylvinylketone (2b), dimethylmalonate (3a), diethylmalonate (3b), dibenzylmalonate (3c), cyclohexanone-2-carboxylic acid ethylester (3e), pivaldehyde (8), and acetophenone (9) were purchased from commercial sources (Acros or AlfaAesar) and stored over 4 Å molecular sieves for 12 h prior to use. Dibenzyl 2-methylmalonate (3d) and benzyl 2-ethyl-3-oxobutanoate (3f) were prepared from dibenzylmalonate and benzyl 3-oxobutanoate from their reaction using NaH (1 equiv) and alkyl halide (methyl iodide or ethyl bromide respectively, 1.1 equiv) in CH₃CN.³⁷ Chalcone derivatives 6b-e were prepared according to literature procedures and recrystallized from EtOH.³⁸ Methoxyamine hydrochloride was purchased from AKScientific, and was used to generate methoxyamine (6). Due to difficulties in obtaining high concentrations of neutralized methoxyamine hydrochloride using KOH and drierite,^{2k} solutions of methoxyamine were generated from the neutralization of methoxyamine hydrochloride with potassium tert-butoxide in minimal THF followed by collection of the distillate at 65° C. RE[N(SiMe₃)₂]₃,³⁹ [M₃(THF)ₙ][(BINOLate)₃RE](M=Li, Na, K)^{5a,5c,8b,8c}, were prepared according to literature procedures.

X-Ray Crystallography.

X-ray intensity data were collected on a Brüker APEXII CCD area detector employing graphite-monochromated Mo—Kα radiation (λ=0.71073 Å) at a temperature of 143(1) K. In all cases, rotation frames were integrated using SAINT,⁴⁰ producing a listing of unaveraged F² and σ(F²) values which were then passed to the SHELXTL⁴¹ program package for further processing and structure solution on a Dell Pentium 4 computer. The intensity data were corrected for Lorentz and polarization effects and for absorption using TWINABS⁴² or SADABS.⁴³ The structures were solved by direct methods (SHELXS-97).⁴⁴ Refinement was by full-matrix least squares based on F² using SHELXL-97. All reflections were used during refinements. Non-hydrogen atoms were refined anisotropically and hydrogen atoms were refined using a riding model. For the structures of [(TMG-H⁺)₃][(BINOLate)₃Eu](1-Eu), [(TMG-H⁺)₃][(BINOLate)₃La].0.5 C₅H₁₂ (1-La.0.5 C₅H₁₂), [(TMG-H⁺)₃][(BINOLate)₃Y].0.5C₅H₁₂ (1-Y.0.5 C₅H₁₂) there were areas of disordered solvent for which reliable disorder models could not be devised; the X-ray data were corrected for the presence of disordered solvent using SQUEEZE.⁴⁵

### Synthetic Details and Characterization

### General Procedure A: Glovebox Synthesis of [TMG-H⁺]₃[(BINOLate)₃RE](1-La.0.5 C₅H₁₂)

Under a dry N₂ atmosphere in a glovebox, a 20 mL glass vial was charged with (S)-BINOL (558.3 mg, 1.95 mmol, 3 equiv; FW: 286.32 g·mol⁻¹), THF (7 mL), and a Teflon-coated stir bar. 1,1,3,3-Tetramethylguanidine (TMG, 245 μL, 1.95 mmol, 3 equiv; FW: 115.18 g·mol⁻¹) was added to the clear stirring colorless solution, and an immediate color change to light yellow was observed. La[N(SiMe₃)₂]₃ (403.1 mg, 0.650 mmol; FW: 620.07 g·mol⁻¹) was added as a solid and stirred for 15 min. The solvent was removed under reduced pressure to yield a crude residue, and the product was crystallized by layering a CH₂Cl₂ solution (4 mL) with pentane (12 mL). After 12-24 h the crystalline solid was isolated by vacuum filtration over a medium porosity frit and dried for 3 h under reduced pressure. Yield: 795 mg (0.578 mmol, 89%; FW: 1376.49 g·mol⁻¹). Anal. Calcd for C_{77.5}H₈₄O₆N₉La: C, 67.63; H, 6.15; N, 9.16. Found: C, 67.59; H, 6.26; N, 8.87. ¹H-NMR (500 MHz, CDCl₃) δ: 8.28 (s, NH₂, 6H), 7.54 (m, 18H), 6.99 (d, J=8.5 Hz, 6H), 6.87 (t, J=7.5 Hz, 6H), 6.80 (t, J=7.2 Hz, 6H), and 1.79 (s, N(CH₃)₂, 36H). ¹³C{¹H}-NMR (126 MHz, CDCl₃) δ: 163.3, 159.7 (H₂N═C), 135.4, 127.5, 127.0, 126.9, 126.6, 125.1, 124.0, 118.9, 118.8, 38.1 (N(CH₃)₂). IR (KBr, cm⁻¹) v: 3421 (br, N—H), 3044, 3028, 2956, 2926, 2903, 2883, 2811, 1687, 1610, 1589, 1567, 1553, 1491, 1461, 1452, 1422, 1407, 1354, 1344, 1284, 1271, 1247, 1238, 1211, 1176, 1147, 1138, 1122, 1068, 1058, 1033, 995, 956, 934, 855, 822, 789, 745, 738, 690, 664, 632, 591, 571, 549, 531, 521, 496, 458. X-ray quality single crystals were obtained from layering concentrated solutions of 1-La in CH₂Cl₂ with pentane (1:4 v/v).

### 1-Ea

The title compound, 1-Eu, was prepared by General Procedure A using Eu[N(SiMe₃)₂]₃ (411.5 mg, 0.650 mmol; FW: 633.13 g·mol⁻¹). Yield: 810 mg (0.598 mmol, 92%; FW: 1353.47 g·mol⁻¹). Anal. Calcd for C₇₅H₇₈O₆N₉Eu: C, 66.56; H, 5.81; N, 9.31. Found: C, 66.22; H, 5.86; N, 8.87. ¹H-NMR (500 MHz, CDCl₃) δ: 18.57 (br s, 6H), 8.07 (d, J=8.5 Hz, 6H), 7.59 (d, J=7.9 Hz, 6H), 7.43 (t, J=7.3 Hz, 6H), 7.20 (t, J=7.3 Hz, 6H), 6.50 (s, 6H), 2.58 (s, Ar—H+N(CH₃)₂, 42H). ¹³C{¹H}-NMR (126 MHz, CDCl₃) δ: 204.0 (H₂N═C), 161.7, 142.5, 128.9, 126.7, 125.6, 125.2, 124.7, 118.5, 118.1, 114.1, 38.9 (N(CH₃)₂). IR (KBr, cm⁻¹) v: 3421 (br, N—H), 3044, 3028, 2956, 2926, 2903, 2883, 2811, 1694, 1610, 1589, 1567, 1552, 1491, 1461, 1452, 1422, 1407, 1354, 1344, 1284, 1271, 1247, 1238, 1211, 1176, 1147, 1138, 1122, 1068, 1052, 1033, 995, 956, 934, 855, 822, 789, 745, 738, 690, 664, 632, 591, 571, 549, 531, 521, 496, 459. X-ray quality single crystals were obtained from layering concentrated solutions of 1-En in THF with pentane (1:4 v/v).

### 1-Yb.0.5 C₅H₁₂

The title compound, 1-Yb, was prepared by General Procedure A using Yb[N(SiMe₃)₂]₃ (425.2 mg, 0.650 mmol; FW: 654.22 g·mol⁻¹). Yield: 828 mg (0.587 mmol, 90%; FW: 1410.64 g·mol⁻¹). Anal. Calcd for Cn_{77.5}H₈₄O₆N₉Yb: C, 65.99; H, 6.00; N, 8.94. Found: C, 65.96; H, 5.75; N, 8.73. ¹H-NMR (500 MHz, CDCl₃) δ: 11.07 (d, J=8.5 Hz, 6H), 9.00 (t, J=8.0 Hz, 6H), 8.54 (d, J=6.5 Hz, 6H), 8.03 (t, J=8.0 Hz, 6H), 5.47 (s, 6H), 4.82 (s, N(CH₃)₂, 36H), −15.69 (s, 6H). The ¹H-NMR resonance corresponding to the NH₂ group was not observed, and is attributed to line broadening from the paramagnetic Yb center. ¹³C{¹H}-NMR (126 MHz, CDCl₃) δ: 171.1, 169.1 (H₂N═C, 147.0, 144.3, 130.6, 129.3, 129.1, 128.6, 125.7, 124.9, 121.3, 41.8 (N(CH₃)₂). IR (KBr, cm⁻¹) v: 3421 (br, N—H), 3044, 3028, 2956, 2926, 2903, 2883, 2811, 1699, 1610, 1589, 1567, 1552, 1491, 1461, 1452, 1422, 1407, 1354, 1344, 1284, 1271, 1247, 1238, 1211, 1176, 1147, 1138, 1122, 1068, 1052, 1035, 995, 956, 935, 855, 822, 789, 745, 738, 690, 664, 632, 591, 571, 549, 531, 521, 496, 460. X-ray quality single crystals were obtained from layering concentrated solutions of 1-Yb in CH₂C₂ with pentane (1:4 v/v).

### 1-Y.0.5 C₅H₁₂

The title compound, 1-Y, was prepared by General Procedure A using Eu[N(SiMe₃)₂]₃ (370.5 mg, 0.650 mmol; FW: 570.07 g·mol⁻¹). Yield: 760 mg (0.573 mmol, 88%; FW: 1326.49 g·mol⁻¹). Anal. Calcd for C_{77.5}H₈₄O₆N₉Y: C, 70.17; H, 6.38; N, 9.50. Found: C, 70.27; H, 6.36; N, 9.16. ¹H-NMR (500 MHz, CDCl₃) δ: 8.14 (br s, NH₂, 6H), 7.57 (d, J=8.7 Hz, 6H), 7.52 (d, J=8.9 Hz, 6H), 7.47 (d, J=7.9 Hz, 6H), 6.98 (d, J=8.4 Hz, 6H), 6.83 (t, J=7.5 Hz, 6H), 6.76 (t, J=7.5 Hz, 6H), 1.73 (s, N(CH₃)₂, 36H). ¹³C{¹H}-NMR (126 MHz, CDCl₃) δ: 164.0, 159.4 (H₂N═C), 135.3, 127.6, 127.5, 126.7, 126.6, 125.1, 123.8, 119.1, 118.8, 38.0 (N(CH₃)₂). IR (KBr, cm⁻¹) v: 3421 (br, N—H), 3044, 3028, 2956, 2926, 2903, 2883, 2811, 1692, 1610, 1589, 1567, 1552, 1491, 1461, 1452, 1422, 1407, 1354, 1344, 1284, 1271, 1247, 1238, 1211, 1176, 1147, 1138, 1122, 1068, 1050, 1035, 995, 956, 935, 855, 822, 789, 745, 738, 690, 664, 632, 591, 571, 549, 531, 521, 496, 463. X-ray quality single crystals were obtained from layering concentrated solutions of 1-Y in CH₂Cl₂ with pentane (1:4 v/v).

### General Procedure B: Open-air Synthesis of [TMG-H⁺]₃[(BINOLate)₃RE](1-RE; RE=La, Eu). (1-La.0.5 C₅H₁₂)

Under ambient atmosphere, a 20 mL glass vial was charged La(NO₃)₃.6H₂O (500 mg, 1.15 mmol, 1 equiv; FW: 433.01 g·mol⁻¹), CH₃CN (10 mL), and a Teflon-coated stir bar. The solution was stirred and (S)-BINOL (990 mg, 3.46 mmol, 3 equiv; FW: 286.32 g·mol⁻¹) was added as a solid. The solution was stirred for ˜5 minutes until all La(NO₃)₃.6H₂O was dissolved. TMG (0.877 mL, 6.93 mmol, 6 equiv; FW: 115.18 g·mol⁻¹) was added via syringe to the clear colorless solution, and immediately formed an off-white precipitate. After ˜1 min of additional stirring, the vial was sealed and centrifuged at 4,000 RPM for 5 min. The supernatant was decanted and the precipitate was dried under reduced pressure on a rotary evaporator. The product was crystallized by layering a concentrated solution of CH₂Cl₂ (4 mL) with pentane (16 mL; 1:4 v/v). After 12-24 h the crystalline solid was isolated by vacuum filtration over a coarse porosity frit and dried for 3 h under reduced pressure (50° C./200 mTorr). Yield: 1.325 g (0.963 mmol, 84%; FW: 1376.49 g·mol⁻¹). ¹H-NMR (500 MHz, CDCl₃) δ: 7.87 (br s, NH₂, 6H), 7.56 (m, 18H), 7.02 (d, J=8.4 Hz, 6H), 6.92 (t, J=7.5 Hz, 6H), 6.86 (t, J=7.2 Hz, 6H), and 1.89 (s, N(CH₃)₂, 36H). ¹³C{¹H}-NMR (126 MHz, CDCl₃) δ: 162.8, 159.9 (H₂N═C), 135.3, 127.5, 127.1, 126.7, 126.6, 125.1, 124.1, 119.0, 118.7, 38.2 (N(CH₃)₂). ¹H-NMR (500 MHz, THF-d) 87.48 (t, J=9.0 Hz, 6H), 7.45 (d, J=8.0 Hz, 6H), 7.41 (d, J=9.0 Hz, 6H), 6.92 (d, J=8.4 Hz, 6H), 6.81 (t, J=8.0 Hz, 6H), 6.76 (t, J=7.0 Hz, 6H), and 1.89 (s, N(CH₃)₂, 36H). ¹³C{¹H}-NMR (500 MHz, THF-d) δ: 164.1 (H₂N═C), 161.3, 136.5, 128.3, 128.0, 127.8, 127.5, 126.0, 124.7, 119.6, 38.7 (N(CH₃)₂).

Alternative Procedure (25 Mmol Scale):

A 500 mL Erlenmeyer flask was charged with La(NO₃)₃.6H₂O (10.83 g, 25.00 mmol, 1 equiv; FW: 433.01 g·mol⁻¹), (S)-BINOL (21.50 g, 75.00 mmol, 3 equiv; FW: 286.32 g·mol⁻¹), CH₃CN (75 mL), and a Teflon-coated stir bar. The solution was stirred for ˜15 minutes until all La(NO₃)₃.6H₂O was dissolved. TMG (18.82 mL, 150.0 mmol, 6 equiv; FW: 115.18 g*mol⁻¹) was added via syringe over 5 min to the clear colorless solution, and immediately formed an off-white precipitate. After 10 min of additional stirring, the precipitate was isolated by vacuum filtration over a coarse porosity frit. After additional drying under reduced pressure on a rotary evaporator, the product was crystallized from a concentrated solution of 1-La in CH₂Cl₂ (˜125 mL) followed by layering with pentane (500 mL; 1:4 v/v). After 12-24 h, the crystalline product was isolated by vacuum filtration over a coarse porosity frit and dried for 3 h under reduced pressure (50° C./200 mTorr). Yield: 24.05 g (17.47 mmol, 70%; FW: 1376.49 g·mol⁻¹).

### 1-Eu

The title compound, 1-Eu, was prepared by General Procedure A using Eu(NO₃)₃.6H₂O (500 mg, 1.12 mmol, 1 equiv; FW: 446.07 g·mol⁻¹). Yield: 1.220 g (0.901 mmol, 80%; FW: 1353.47 g·mol⁻¹). ¹H-NMR (500 MHz, CDCl₃) δ: 14.03 (br s, H₂NH₂ 6H), 7.87 (s, 6H), 7.54 (s, 6H), 7.34 (s, 6H), 7.11 (s, 6H), 6.57 (s, 6H), 3.23 (s, 6H), 2.57 (s, N(CH₃)₂, 36H). ¹H-NMR (500 MHz, THF-d₈) δ: 18.00 (br s, NH₂, 6H), 7.95 (d, J=8.2 Hz, 6H), 7.46 (d, J=8.3 Hz, 6H), 7.30 (t, J=7.5 Hz, 6H), 7.07 (t, J=7.7 Hz, 6H), 6.39 (d, J=6.6 Hz, 6H), 2.64 (d, J=6.0 Hz, 6H), 2.51 (s, N(CH₃)₂, 36H).

### General Procedure C: Benchtop Synthesis of [TMG-H+]₃[(BINOLate)RE](1-RE; RE=Y, Yb). 1-Yb.0.5 C₅H₁₂

Under ambient atmosphere, a 20 mL glass vial was charged Yb(NO₃)₃.6H₂O (500 mg, 1.07 mmol, 1 equiv; FW: 467.15 g·mol⁻¹), CH₃CN (4 mL), glacial acetic acid (185 μL, 3.21 mmol, 3 equiv; FW: 60.05 g·mol⁻¹), and a Teflon-coated stir bar. The solution was stirred for 5 min and a solution of(S)-BINOL (919.4 mg, 3.21 mmol, 3 equiv; FW: 286.32 g·mol⁻¹) and TMG (0.805 mL, 6.42 mmol, 6 equiv; FW: 115.18 g·mol⁻¹) in CH₃CN (3 mL) was added dropwise over 2 min. Upon completion of the addition a small amount of precipitate had formed, and TMG (0.405 mL, 3.21 mmol, 3 equiv; FW: 115.18 g·mol⁻¹) was added dropwise, and immediately formed an off-white precipitate. After ˜1 min of additional stirring, the vial was sealed and centrifuged at 4,000 RPM for 5 min. The supernatant was decanted and the precipitate was dried under reduced pressure on a rotary evaporator. The product was crystallized by layering a concentrated solution of CH₂Cl₂ (6 mL) with pentane (24 mL; 1:4 v/v). After 12-24 h the crystalline solid was isolated by vacuum filtration over a coarse porosity frit and dried for 3 h under reduced pressure (50° C./200 mTorr). Yield: 1.250 g (0.886 mmol, 83%; FW: 1410.64 g·mol⁻¹). ¹H-NMR (500 MHz, CDCl₃) δ: 10.60 (s, 6H), 8.73 (s, 6H), 7.84 (s, 6H), 5.60 (s, 611), 3.68 (s, N(CH₃)₂, 36H), −13.87 (s, 6H). The ¹H-NMR resonance corresponding to the TMG-H⁺ NH₂ group was not observed, and is attributed to line broadening from the paramagnetic Yb center.

6.5 mmol scale; 1-Y.0.5 C₅H₁₂: A 125 mL Erlenmeyer flask was charged with Y(NO₃)₃.6H₂O (2.500 g, 6.53 mmol, 1 equiv; FW: 383.01 g·mol⁻¹), CH₃CN (20 mL), glacial acetic acid (1.12 mL, 19.58 mmol, 3 equiv; FW: 60.05 g·mol⁻¹), and a Teflon-coated stir bar. The clear colorless solution was stirred for 5 min and a solution of(S)-BINOL (4.510 g, 19.6 mmol, 3 equiv; FW: 286.32 g·mol⁻¹) and TMG (4.91 mL, 39.2 mmol, 6 equiv; FW: 115.18 g·mol⁻¹) in CH₃CN (10 mL) was added dropwise over 20 min. Upon completion of the addition a small amount of precipitate had formed. Additional TMG (2.46 mL, 19.6 mmol, 3 equiv; FW: 115.18 g·mol⁻¹) was added dropwise, and immediately formed an off-white precipitate. The precipitate was isolated by vacuum filtration over a coarse porosity frit. After additional drying under reduced pressure on a rotary evaporator, the product was crystallized from a concentrated solution of 1-Y in CH₂Cl₂ (˜25 mL) followed by layering with pentane (100 mL; 1:4 v/v). After 12-24 h, the crystalline product was isolated by vacuum filtration over a coarse porosity frit and dried for 3 h under reduced pressure (50° C./200 mTorr). Yield: 7.11 g (5.36 mmol, 82%; FW: 1326.49 g·mol⁻¹). ¹H-NMR (500 MHz, CDCl₃) δ: 8.11 (br s, NH₂, 6H), 7.61 (d, J=9.2 Hz, 6H), 7.53 61 (d, J=9.2 Hz, 6H), 7.51 61 (d, J=9.2 Hz, 6H), 6.90 (d, J=9.2 Hz, 3H), 6.83 (d, J=9.2 Hz, 3H), 6.81 (d, J=9.2 Hz, 3H), 1.84 (s, N(CH₃)₂, 36H). ¹³C{¹H}-NMR (126 MHz, CDCl₃) δ: 163.9, 159.5 (H₂N═C), 135.2, 127.5, 127.4, 126.7, 126.6, 125.1, 123.8, 119.1, 118.7, 38.1 (N(CH₃)₂).

NMR-Scale Generation of LaLB from 1-La and LiI.

An NMR tube was charged with 1-La.0.5 C₅H₁₂ (15.0 mg, 0.0109 mmol, 1 equiv; FW: 1376.49 g·mol⁻¹) and THF-d₈ (0.50 mL). LiI (4.4 mg, 0.0329 mmol, 3.0 equiv; FW: 133.85 g·mol⁻¹) was added to the clear colorless solution, which resulted in the immediate precipitation of tetramethylguanidinium iodide ([TMG-H⁺][I⁻]) and a color change to pale light yellow. ¹H-NMR (400 MHz, THF-d₈) δ: 7.61 (m, 12H), 7.06 (d, J=8.4 Hz, 6H), 6.89 (t, J=6.0 Hz, 6H), 6.80 (m, 12H). ⁷Li{¹H}-NMR(155 Hz, THF-d₈) δ: −2.0. ¹H-NMR spectra was consistent with the previously reported data.^{8b, 15}

NMR-Scale Generation of LaNaB from 1-La and NaI.

An NMR tube was charged with 1-La-0.5 C₅H₁₂ (15.0 mg, 0.0109 mmol, 1 equiv; FW: 1376.49 g·mol⁻¹) and THF-d₈ (0.50 mL). NaI (4.9 mg, 0.0329 mmol, 3.0 equiv; FW: 133.85 g·mol⁻¹) was added to the clear colorless solution, which resulted in the immediate precipitation of tetramethylguanidinium iodide ([TMG-H⁺][I⁻]) and a color change to pale light yellow. ¹H-NMR (400 MHz, THF-d₈) δ: 7.55 (d, J=9.2 Hz, 6H), 7.49 (t, J=6.6 Hz, 6H), 7.37 (d, J=8.8 Hz, 6H), 6.84 (m, 6H), 6.79 (m 12H). ¹H-NMR spectra was consistent with the previously reported data.^{4,8a}

NMR-Scale Generation of LaKB from 1-La and KO^{t}Bu.

An NMR tube was charged with 1-La.0.5 C₅H₁₂ (15.0 mg, 0.0109 mmol, 1 equiv; FW: 1376.49 g·mol⁻¹) and THF-d₈ (0.50 mL). KO^{t}Bu (3.7 mg, 0.0329 mmol, 3.0 equiv; FW: 112.21 g·mol⁻¹) was added to the clear colorless solution, and resulted in an immediate color change to pale light yellow. ¹H-NMR (400 MHz, THF-d₈) δ: 7.59 (d, J=8.8 Hz, 6H), 7.52 (d, J=7.8 Hz, 6H), 7.31 (d, J=8.8 Hz, 6H), 6.77 (m, 18H).

NMR-Scale Generation of EuLB from 1-En and LiI.

An NMR tube was charged with 1-En (15.0 mg, 0.0111 mmol, 1 equiv; FW: 1353.47 g·mol⁻¹) and THF-ds (0.50 mL). LiI (6.7 mg, 0.0499 mmol, 4.5 equiv; FW: 133.85 g·mol⁻¹) was added to the clear colorless solution, which resulted in the immediate precipitation of tetramethylguanidinium iodide ([TMG-H⁺][I⁻]) and a color change to pale light yellow. ¹H-NMR (400 MHz, THF-d₈) δ: 24.39 (br s, 6H), 10.67 (s, 6H), 7.49 (s, 6H), 6.24 (s, 6H), 4.11 (s, 6H), 0.94 (s, 6H). ⁷Li{H}-NMR(155 Hz, THF-d₈) δ: 39.0. ¹H-NMR spectra was consistent with the previously reported data.^{4,5b,5c}

NMR-Scale Generation of YLB from 1-Y and LiI.

An NMR tube was charged with 1-Y.0.5 C₅H₁₂ (15.0 mg, 0.0113 mmol, 1 equiv; FW: 1326.49 g·mol⁻¹) and THF-ds (0.50 mL). LiI (6.8 mg, 0.0509 mmol, 4.5 equiv; FW: 133.85 g·mol⁻¹) was added to the clear colorless solution, which resulted in the immediate precipitation of tetramethylguanidinium iodide ([TMG-H⁺][I⁻]) and a color change to pale light yellow. ¹H-NMR (400 MHz, THF-d₈) δ: 7.63 (br s, 12H), 7.32 (br s, 6H), 6.82 (m, 18H). ⁷Li{¹H}-NMR(155 Hz, THF-d₈) δ: 0.88.

### General Procedure D: Asymmetric Michael addition of symmetrical malonates (3a-d) to cyclohexenone (2a). (S)-3-[bis(methoxycarbonyl)methyl]cyclohexanone (4a)

Under an N₂ flow on a Schlenk line, a 10 mL Schlenk flask was charged with 1-La (134 mg, 0.0973 mmol, 10 mol %, FW: 1376.49 g·mol⁻¹), NaI (45.0 mg, 0.300 mmol, 30 mol %, FW: 149.89 g·mol⁻¹), THF (2.0 mL), and a Teflon-coated stir-bar. H₂O (5.40 μL, 0.300 mmol, 30 mol %, FW: 18.02 g·mol⁻¹) was added to the pale light yellow mixture. Cyclohexenone (2a, 97.0 μL, 1.00 mmol, 1 equiv; FW: 96.1 g·mol⁻¹) was added, resulting in an immediate color change to dark yellow. The reaction vessel was sealed with a 14/20 rubber septum, wrapped with parafilm, and cooled to 0° C. Dimethylmalonate (3a, 114.5 μL, 1.00 mmol, 1 equiv; FW: 132.12 g·mol⁻¹) was added in 4 portions over 20 min. After 12 h, the reaction was quenched with HCl (10% v/v, 2 mL) and extracted with CH₂Cl₂ (3×10 mL). The organic layers were combined, washed with brine (10 mL), dried with MgSO₄, filtered, and solvent was removed under reduced pressure. The crude residue was purified by column chromatography (SiO₂, 33% Pet. ether:Et₂O) to obtain 4a as a colorless oil. Yield: 202.4 mg. (0.887 mmol, 89% yield, 94% ee; FW: 228.22 g·mol⁻¹). Enantioselectivities were determined by HPLC: Chiralcel, AS-H, 10% ^{t}PrOH:hexanes, 1.0 mL-min⁻¹, λ_{obs}=210 nm, t_{R}=16.67, 18.73 min. The ¹H- and ¹³C{¹H}-NMR spectra match the previously reported spectra.^{2c}

2.5 Mol % [La] Loading (8.7 Mmol Scale):

Under an N₂ flow on a Schlenk line, a 10 mL Schlenk flask was charged with 1-La (300 mg, 0.218 mmol, 2.5 mol %, FW: 1376.49 g·mol⁻¹), NaI (98.0 mg, 0.654 mmol, 7.5 mol %, FW: 149.89 g·mol⁻¹), dry THF (2.2 mL), and a Teflon-coated stir-bar. H₂O (11.80 μL, 0.654 mmol, 7.5 mol %, FW: 18.02 g·mol⁻¹) was added to the pale light yellow mixture. Cyclohexenone (2a, 0.845 mL, 8.70 mmol, 1 equiv; FW: 96.1 g·mol⁻¹) was added, followed by an immediate color change to dark yellow. The reaction vessel was sealed with a 14/20 rubber septum, wrapped with parafilm, and cooled to 0 C. Dimethylmalonate (3a) was added via syringe pump over 8 h. After a total of 24 h, the reaction was quenched with HCl (10% v/v, 2 mL) and extracted with CH₂C₂ (3×10 mL). The organic layers were combined, washed with brine (10 mL), dried over MgSO₄, filtered, and the solvent was removed under reduced pressure to yield crude 4a. Yield: 1.850 g. (0.811 mmol, 93% yield, 89% ee; FW: 228.22 g·mol⁻¹). 4a could be purified via crystallization from Et₂O:hexanes (1:3 v/v) at −30° C. Yield: 1.75 g. (7.67 mmol, 88% yield, 94% ee; FW: 228.22 g·mol⁻¹).

Initial High-Throughput Experimentation Optimization of 1-RE/NaX (25.0 μMol Scale):

A 96-well aluminum block containing 1 mL glass vials was dosed with 1-La (2.5 μmol) in CH₂Cl₂ (100 μL), NaX sources (7.5 mol) in THF (100 μL), and the solvent was removed by using a GeneVac. A parylene stir bar was added to each reaction vial, along with cyclohexenone (2a) and dibenzylmalonate (3c) in the desired solvent (50 μL). The 96-well plate was then sealed and stirred for 12 h at RT. The plate was then opened to air and then acetonitrile (500 μL) was added to each vial. The plate was covered and stirred for 5 min followed by a 5 min period to allow insoluble particulate to settle. Into a separate 96-well LC block, acetonitrile (700 L) and sample (50 μL) were added. The LC block was sealed with a silicon-rubber storage mat and mounted on an automated SFC instrument for analysis using an AS-H column (gradient: 10%→30%→10% IPA: SC—CO₂ (10 min total)). Conditions investigated over several screens (24 and 96-well plates) include: NaX source (X: Cl, Br, I, BF₄, PF₆, OTf, B(Ar)₄, N(SiMe₃)₂, O^{t}Bu, CN, CO₃²⁻), solvent (THF, toluene), water (0, 10 mol %), amount NaX (0, 10, 20, 30, 60 mol %).

### (S)-3-[bis(ethoxycarboayl)methyl]cyclohexenone (4b)

The title compound, 4b, was prepared using General Procedure D using 1-La (134 mg, 0.0973 mmol, 10 mol %, FW: 1376.49 g·mol⁻¹), NaI (45.0 mg, 0.300 mmol, 30 mol %, FW: 149.89 g·mol⁻¹), THF (2.0 mL), H₂O (5.40 μL, 0.300 mmol, 30 mol %, FW: 18.02 g·mol⁻¹), 2a (97.0 μL, 1.00 mmol, 1 equiv; FW: 96.1 g·mol⁻¹), and 3b (153 μL, 1.00 mmol, 1 equiv; FW: 160.17 g·mol⁻¹). 4b was purified by column chromatography (SiO₂, 30% acetone:hexanes) to yield a colorless oil. Yield: 231.2 mg. (0.902 mmol, 90% yield, 96% ee; FW: 256.27 g·mol⁻¹). Enantioselectivities were determined by HPLC: Chiralcel, AS-H, 10% ⁱPrOH:hexanes, 1.0 mL·min⁻¹, λ_{obs}=220 nm, t_{R}=10.01, 10.85 min. The ¹H- and ¹³C{¹H}-NMR spectra match the previously reported spectra.^{2c}

### (S)-3-[bis(benzyloxycarbonyl)methyl]cyclobexenone (4c)

The title compound, 4c, was prepared using General Procedure D using 1-La (134 mg, 0.0973 mmol, 10 mol %, FW: 1376.49 g·mol⁻¹), NaI (45.0 mg, 0.300 mmol, 30 mol %, FW: 149.89 g·mol⁻¹), THF (2.0 mL), H₂O (5.40 μL, 0.300 mmol, 30 mol %, FW: 18.02 g·mol⁻¹), 2a (97.0 μL, 1.00 mmol, 1 equiv; FW: 96.1 g·mol⁻¹), and 3b (250 μL, 1.00 mmol, 1 equiv; FW: 284.31 g·mol⁻¹). 4c was purified by column chromatography (SiO₂, 25% acetone:hexanes) to yield a white solid. Yield: 358.6 mg. (0.943 mmol, 94% yield, 88% ee; FW: 380.41 g·mol⁻¹). Enantioselectivities were determined by HPLC: Chiralcel, AS-H, 10% ⁱPrOH:hexanes, 1.0 mL·min⁻¹, λ_{obs}=210 nm, t_{R}=16.54, 18.72 min. The ¹H- and ¹³C{¹H}-NMR spectra match the previously reported spectra.^{2c}

### (S)-3-[bis(benzyloxycarbonyl)ethyl]cyclohexenone (4d)

The title compound, 4d, was prepared using General Procedure D using 1-La (44.9 mg, 0.0326 mmol, 10 mol %, FW: 1376.49 g·mol⁻¹), NaI (14.7 mg, 0.0978 mmol, 30 mol %, FW: 149.89 g·mol⁻¹), THF (0.60 mL), H₂O (1.76 μL, 0.0978 mmol, 30 mol %, FW: 18.02 g·mol⁻¹), 2a (32.2 μL, 0.335 mmol, 1 equiv; FW: 96.1 g·mol⁻¹), and 3d (86.2 μL, 0.335 mmol, 1 equiv; FW: 160.17 g·mol⁻¹), where 3d was added as a solution in THF (0.100 mL). 4d was purified by column chromatography (SiO₂, 25% acetone:hexanes) to yield a pale light yellow oil. Yield: 114.6 mg. (0.291 mmol, 87% yield, 96% ee; FW: 394.43 g·mol⁻¹). Enantioselectivities were determined by HPLC: Chiralcel, AS-H, 10% ^{t}PrOH:hexanes, 1.0 mL·min⁻¹, λ_{obs}=254 nm, t_{R}=11.90, 17.90 min. The ¹H- and ¹³C{¹H}-NMR spectra match the previously reported spectra.^{2c}

### General Procedure E: Asymmetric Michael addition of beta-ketoesters (3e-f) to methyl vinyl ketone (2b). Ethyl (S)-2-oxo-1-(3-oxobutyl)-cyclohexanecarboxylate (4e)

Under an N₂ flow on a Schlenk line, a 10 mL Schlenk flask was charged with 1-La (67 mg, 0.0489 mmol, 5 mol %, FW: 1376.49 g·mol⁻¹), NaI (22.0 mg, 0.147 mmol, 15 mol %, FW: 149.89 g·mol⁻¹), dry THF (1.0 mL), and a Teflon-coated stir-bar. Upon mixing, the immediate formation of a precipitate was observed ([TMG-H⁺][I⁻]). After 1 min, the solvent was removed under reduced pressure (Schlenk line, 30 min), and dry CH₂Cl₂ (2.0 mL) and H₂O (2.64 μL, 0.147 mmol, 15 mol %, FW: 18.02 g·mol⁻¹) were added. The reaction vessel was cooled to −50° C., and methyl vinyl ketone (2b, 100 μL, 1.20 mmol, 1.2 equiv; FW: 70.09 g·mol⁻¹) was added. Cyclohexyl ethyl ester (3e, 160.0 μL, 1.00 mmol, 1 equiv; FW: 170.21 g·mol⁻¹) was added via syringe pump over 8 h. After a total of 20 h, the reaction was quenched by passing the reaction through a short plug of SiO₂ (˜100 mg, in a pipet), which was rinsed with acetone (5 mL). Solvent was removed under reduced pressure, and the crude residue was purified by column chromatography (SiO₂, 20% acetone:hexanes) to yield 4e as a colorless oil. Yield: 210.5 mg. (0.876 mmol, 88% yield, 98% ee; FW: 240.30 g·mol⁻¹). Enantioselectivities were determined by HPLC: Chiralcel, AS-H, 10% ⁱPrOH:hexanes, 1.0 mL·min⁻¹, λ_{obs}=210 nm, t_{R}=9.64, 11.03 min. The ¹H- and ¹³C{¹H}-NMR spectra match the previously reported spectra.⁴⁶

### Benzyl (S)-2-acetyl-2-ethyl-5-oxohexanoate (4f)

The title compound, 4f, was prepared using General Procedure E using with 1-La (67 mg, 0.0489 mmol, 5 mol %, FW: 1376.49 g·mol⁻¹), NaI (22.0 mg, 0.147 mmol, 15 mol %, FW: 149.89 g·mol⁻¹), dry CH₂Cl₂ (2.0 mL) and H₂O (2.64 μL, 0.147 mmol, 15 mol %, FW: 18.02 g·mol⁻¹), Methyl vinyl ketone (2b, 100 μL, 1.20 mmol, 1.2 equiv; FW: 70.09 g·mol⁻¹), and benzyl-2-ethyl-3-oxobutanoate (3e, 206 μL, 1.00 mmol, 1 equiv; FW: 220.26 g·mol⁻¹). 4f was purified by column chromatography (SiO₂, 20% acetone:hexanes) to yield a pale light yellow oil. Yield: 242.5 mg. (0.835 mmol, 84% yield, ≧99% ee; FW: 290.35 g·mol⁻¹). HRMS (ESI) m/z C₁₇H₂₂O₄Na, [4f+Na⁺]: Calcd=313.1416. Found=313.1405. [α]_{D}²⁰=−7.947 (c=2.932, CHCl₃). ¹δ 7.32 (s, 5H), 5.14 (s, 1H), 5.13 (s, 1H), 2.22 (dt, J=8.2, 6.1 Hz, 2H), 2.18-2.09 (m, 1H), 2.08-2.00 (m, 1H) 2.04 (s, 3H), 2.02 (s, 3H), 1.97-1.79 (m, 2H), 0.74 (t, J=7.5 Hz, 3H).¹³207.1, 204.9, 172.1, 135.4, 128.7, 128.6, 67.1, 63.1, 38.3, 29.9, 26.9, 25.3, 24.8, 8.3. IR (neat, cm⁻¹) v: 3066, 3035, 2965, 2925, 2883, 2856, 1737, 1711, 1606, 1587, 1497, 1456, 1420, 1373, 1356, 1279, 1239, 1208, 1166, 1122, 1099, 1064, 1030, 969, 912, 827, 794, 752, 699, 602, 584, 516, 497, 457. Enantioselectivities were determined by HPLC: Chiralcel, AD-H, 1% ⁱPrOH:hexanes, 0.5 mL·min⁻¹, λ_{obs}=220 nm, t_{R}=53.84, 57.96 min.

### General Procedure F: Asymmetric aza-Michael addition methoxyamine (6) to chalcone derivatives (5). (S)-3-(Methoxyamino)-1,3-diphenyl-1-propanone (7a)

A microwave vial was charged with 1-La (20.6 mg, 0.0150 mmol, 3 mol %, FW: 1376.49 g·mol⁻¹), LiI (6.0 mg, 0.045 mmol, 9 mol %, FW: 133.85 g·mol⁻¹), Chalcone (104.1 mg, 0.500 mmol, 1 equiv; FW: 208.26 g·mol⁻¹), Drierite® (68.1 mg, 0.5 mmol, 1 equiv; FW: 136.14), and a Teflon-coated stir-bar. The vessel was sealed with a 14/20 rubber septum, and evacuated and refilled with N₂ three times. Dry THF (0.250 mL) was added and the immediate formation of a precipitate was observed ([TMG-H⁺][I⁻]). The stirring orange mixture was cooled to −20° C. and methoxyamine (6, 56.6 μL, 0.600 mmol, 10.6 M in THF, 1.2 equiv; FW: 47.06 g·mol⁻¹) was added via syringe and the reaction was stirred for 48 h under N₂. Acetaldehyde (15.0 μL, 0.268 mmol, 0.44 equiv; FW: 44.05 g·mol⁻¹) was added to quench excess methoxyamine. The reaction was diluted with diethyl ether (5 mL), washed with water (3×5 mL), brine (5 mL), and dried with MgSO₄. Solvents were removed under reduced pressure, and the crude residue was purified by column chromatography (SiO₂, 10% EtOAc:hexanes) to yield 7a as a light yellow solid. Yield: 112.3 mg. (0.440 mmol, 88% yield, 91% ee; FW: 255.32 g·mol⁻¹). Enantioselectivities were determined by HPLC: Chiralcel, OD-H, 5% ⁱPrOH:hexanes, 0.5 mL·min⁻¹, λ_{obs}=254 nm, t_{R}=21.39, 28.82 min. The ¹H- and ¹³C{¹H}-NMR spectra match the previously reported spectra.^{2k,33}

7a (5 Mmol Scale).

A 10 mL Schlenk flask was charged with 1-La (206.5 mg, 0.150 mmol, 3 mol %, FW: 1376.49 g·mol⁻¹), LiI (60.2 mg, 0.450 mmol, 9 mol %, FW: 133.85 g·mol⁻¹), and a Teflon-coated stir-bar and purged with N₂. Dry THF (2.50 mL) was added and the immediate formation of a precipitate was observed ([TMG-H⁺][I⁻]). Chalcone (1.041 g, 5.00 mmol, 1 equiv; FW: 208.26 g·mol⁻¹) was added against an N₂ flow to the stirring light yellow mixture. Drierite (680.7 mg, 5.0 mmol, 1 equiv; FW: 136.14 g·mol⁻¹) was added against an N₂ flow to the stirring orange mixture. The reaction vessel was cooled to −20° C. and 6 (0.566 mL, 6.00 mmol, 10.6 M in THF, 1.2 equiv; FW: 47.06 g·mol⁻¹) was added via syringe and the reaction was stirred for 48 h. Acetaldehyde (0.150 mL, 2.68 mmol, 0.44 equiv; FW: 44.05 g·mol⁻¹) was added to quench excess methoxyamine. The reaction was diluted with diethyl ether (15 mL), washed with water (3×10 mL), brine (10 mL), and dried with MgSO₄. The solvents was removed under reduced pressure, and the crude residue was purified by column chromatography (SiO₂, 10% EtOAc:hexanes) to yield 7a as a light yellow solid. Yield: 1.187 g. (4.65 mmol, 93% yield, 93% ee; FW: 255.32 g·mol⁻¹).

### 3-(Methoxyamino)-3-(4-methylphenyl)-1-phenyl-1-propanone (7b)

The title compound, 7b, was synthesized following General Procedure F using (E)-1-phenyl-3-(p-tolyl)prop-2-en-1-one (111.2 mg, 0.500 mmol, 1 equiv; FW: 222.29 g·mol⁻¹) for 50 h. Yield: 122.5 mg. (0.455 mmol, 91% yield, 93% ee; FW: 269.34 g·mol⁻¹). Enantioselectivities were determined by HPLC: Chiralcel, OD-H, 5% ⁱPrOH:hexanes, 1.0 mL·min⁻¹, λ_{obs}=280 nm, t_{R}=8.08, 11.79 min. The ¹H- and ¹³C{¹H}-NMR spectra match the previously reported spectra.^{2k,33}

### 1-(4-Chlorophenyl)-3-(methoxyamino)-3-phenyl-1-propanone (7c)

The title compound, 7c, was synthesized following General Procedure F using (E)-1-(4-chlorophenyl)-3-phenylprop-2-en-1-one (121.4 mg, 0.500 mmol, 1 equiv; FW: 242.70 g·mol⁻¹) for 50 h. Yield: 139.1 mg. (0.480 mmol, 96% yield, 94% ee; FW: 289.76 g·mol⁻¹). Enantioselectivities were determined by HPLC: Chiralcel, AD-H, 5% ⁱPrOH:hexanes, 0.7 mL·min⁻, λ_{obs}=210 nm, t_{R}=25.13, 27.13 min. The ¹H- and ¹³C{¹H}-NMR spectra match the previously reported spectra.^{2k,33}

### 3-(Methoxyamino)-1-phenyl-3-(2-thienyl)-1-propanone (7d)

The title compound, 7d, was synthesized following General Procedure F using (E)-1-phenyl-3-(thiophen-2-yl)prop-2-en-1-one (107.1 mg, 0.500 mmol, 1 equiv; FW: 214.28 g·mol⁻¹) for 48 h. Yield: 121.5 mg. (0.465 mmol, 93% yield, 93% ee; FW: 261.34 g·mol⁻¹). Enantioselectivities were determined by HPLC: Chiralcel, OD-H, 5% ⁱPrOH:hexanes, 1.0 mL·min⁻¹, λ_{obs}=254 nm, t_{R}=11.62, 18.29 min. The ¹H- and ¹³C{H}-NMR spectra match the previously reported spectra.³

### 3-(Methoxyamino)-1,5-diphenyl-4-penten-1-one (7e)

The title compound, 7e, was synthesized following General Procedure F using (2E,4E)-1,5-diphenylpenta-2,4-dien-1-one (117.2 mg, 0.500 mmol, 1 equiv; FW: 234.34 g·mol⁻¹) and THF (0.250 mL) for 90 h. Yield: 136.5 mg. (0.485 mmol, 97% yield, 91% ee; FW: 281.35 g·mol⁻¹). Enantioselectivities were determined by HPLC: Chiralcel, OD-H, 5% ⁱ PrOH:hexanes, 0.5 mL·min⁻¹, =230 nm, t_{R}=26.55, 32.24 min. The ¹H- and ¹³C{¹H}-NMR spectra match the previously reported spectra.^{2k,33}

### 3-Hydroxy-4,4-dimethyl-1-phenylpentan-1-one (10)

Under an N₂ flow, a 10 mL Schlenk flask was charged with dry THF (0.3 mL) and KO^{t}Bu (9.0 mg, 0.0800 mmol, 8 mol %, FW: 112.21 g·mol⁻¹). H₂O (2.88 μL, 0.160 mmol, 16 mol %, FW: 18.02 g·mol⁻¹) was added. A dry 4 mL scintillation vial was charged with 1-La (67 mg, 0.0489 mmol, 8 mol %, FW: 1376.49 g·mol⁻¹), LiI (22.0 mg, 0.147 mmol, 24 mol %, FW: 133.85 g·mol⁻¹), dry THF (1.2 mL), and a Teflon-coated stir-bar. The immediate formation of a precipitate was observed ([TMG-H⁺][I⁻]). The solution was immediately transferred to the Schlenk flask and cooled to −20° C. Acetophenone (8, 0.584 mL, 5.00 mmol, 5 equiv; FW: 120.15 g·mol⁻¹) was added via syringe and stirred for 20 min. Pivaldehyde (9, 108.6 μL, 1.00 mmol, 1 equiv; FW: 86.13 g·mol⁻¹) was added and stirred for 20 h. The reaction was quenched with HCl (1 N, 1 mL), extracted with Et₂O (3×15 mL) and washed with water (5 mL). The compound was dried over MgSO₄, filtered, and solvent was removed under reduced pressure. The crude oil was purified by column chromatography (SiO₂, 5% EtOAc:hexanes) to yield 10 as a colorless oil. Yield: 152.0 mg. (0.736 mmol, 74% yield, 95% ee; FW: 206.28 g·mol⁻¹). Enantioselectivities were determined by HPLC: Chiralcel, AD-H, 15% ⁱ PrOH:hexanes, 1.0 mL·min⁻¹, λ_{obs}=254 nm, t_{R}=4.68, 5.93 min. The ¹H- and ¹³C{¹H}-NMR spectra match the previously reported spectra.⁴⁷

Among the advantages of the precatalysts of this invention are the following:

1. Hydrogen bonding provides a well-defined crystalline rare earth BIONLate complexes;

2. Acid-base cation exchange generates REMB species;

3. Selectivity can be influenced by the coordination of the ammonium conjugate base;

4. Alternative M=X allows facile generation of REMB and innocent spectator ions; and

5. Complex self assembly is dictated by choice of cation (ammonium).

As those skilled in the art will appreciate, the present invention provides a straightforward, high-yielding, and scalable open-air syntheses that enable rapid access to crystalline, non-hygroscopic complexes from inexpensive hydrated RE starting materials. The resulting complexes can be used as precatalysts for Shibasaki's REMB frameworks, having demonstrated comparable or improved levels of stereoselectivity in several mechanistically diverse reactions.

Use of hydrated RE sources provides a significant cost reduction; RE(NO₃)₃.XH₂O are ˜100 fold cheaper than commonly employed functionalized RE materials such as RE(OⁱPr)₃ or RE[N(SiMe₃)₂]₃.³⁵ Due to these properties, 1-RE were identified as excellent precursors for the generation of anhydrous heterobimetallic complexes by acid-base or cation-exchange methods with a variety of RE/M combinations.

Furthermore, the present inventors have demonstrated that 1-RE/MI could be applied as a general precatalyst system for Shibasaki's REMB framework using both traditional bench-scale and HTE techniques. This precatalyst system shows comparable or improved performance to the reported REMB systems, and is amenable to different RE/M combinations, different reaction types (Lewis-acid/Brønsted-base, Lewis-acid/Lewis-acid), and the presence of additives. We attribute the success of this particular system to the use of MI, which cleanly generates REMB through cation-exchange while producing an innocent guanidinium iodide spectator-ion. We expect that this system will provide a convenient and complementary synthetic strategy to well-known, and as of yet, unidentified heterobimetallic frameworks. Further investigations on the self-assembly of ionic H-bond pairs, identification of new heterobimetallic frameworks through cation-exchange, and their applications in catalysis are underway.

While certain embodiments of the present invention have been described and/or exemplified above, various other embodiments will be apparent to those skilled in the art from the foregoing disclosure. The present invention is, therefore, not limited to the particular embodiments described and/or exemplified, but is capable of considerable variation and modification without departure from the scope of the appended claims.

A number of publications and patent documents are cited throughout the specification in order to describe the state of the art to which this invention pertains. Each of these citations is incorporated by reference herein as though set forth in full.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. The supported, mixed metal oxide catalyst, its methods of preparation and use can in alternate embodiments, be more specifically defined by any of the transitional terms “comprising”, “consisting essentially of” and “consisting of”.

### REFERENCES

1. (a) Shibasaki, M.; Sasai, H.; Arai, T., Angew. Chem. Int. Ed. 1997, 36 (12), 1237-1256. (b) Shibasaki, M.; Yoshikawa, N., Chem. Rev. 2002, 102 (6), 2187-2209. (c) Jacobsen, E. N.; Pfaltz, A.; Yamamoto, H., Comprehensive Asymmetric Catalysis. Springer: New York, 2004. (d) Shibasaki, M.; Kanai, M.; Matsunaga, S.; Kumagai, N., Acc. Chem. Res. 2009, 42 (8), 1117-1127. (e) Walsh, P. J.; Kozlowski, M. C., Funadmentals of Asymmetric Catalysis. University Science Books: Sausalito, C A, 2008; p 674. (f) Park, J.; Hong, S., Chem. Soc. Rev. 2012, 41 (21), 6931-6943.
2. (a) Sasai, H.; Suzuki, T.; Itoh, N.; Tanaka, K.; Date, T.; Okamura, K.; Shibasaki, M., J. Am. Chem. Soc. 1993, 115 (22), 10372-10373. (b) Shibasaki, M.; Sasai, H., J. Synth. Org. Chem. Jpn. 1993, 51 (11), 972-984. (c) Sasai, H.; Arai, T.; Satow, Y.; Houk, K. N.; Shibasaki, M., J. Am. Chem. Soc. 1995, 117 (23), 6194-6198. (d) Sasai, H.; Arai, S.; Tahara, Y.; Shibasaki, M., J. Org. Chem. 1995, 60 (21), 6656-6657. (e) Sasai, H.; Bougauchi, M.; Arai, T.; Shibasaki, M., Tetrahedron Lett. 1997, 38 (15), 2717-2720. (f) Morita, T.; Arai, T.; Sasai, H.; Shibasaki, M., Tetrahedron-Asymmetry 1998, 9 (8), 1445-1450. (g) Emori, E.; Arai, T.; Sasai, H.; Shibasaki, M., J. Am. Chem. Soc. 1998, 120 (16), 4043-4044. (h) Groger, H.; Saida, Y.; Sasai, H.; Yamaguchi, K.; Martens, J.; Shibasaki, M., J. Am. Chem. Soc. 1998, 120 (13), 3089-3103. (i) Yoshikawa, N.; Yamada, Y. M. A.; Das, J.; Sasai, H.; Shibasaki, M., J. Am. Chem. Soc. 1999, 121 (17), 4168-4178. (j) Schlemminger, I.; Saida, Y.; Groger, H.; Maison, W.; Durot, N.; Sasai, H.; Shibasaki, M.; Martens, J., J. Org. Chem. 2000, 65 (16), 4818-4825. (k) Yamagiwa, N.; Qin, H. B.; Matsunaga, S.; Shibasaki, M., J. Am. Chem. Soc. 2005, 127 (38), 13419-13427. (1) Yamagiwa, N.; Tian, J.; Matsunaga, S.; Shibasaki, M., J. Am. Chem. Soc. 2005, 127 (10), 3413-3422. (m) Sone, T.; Yamaguchi, A.; Matsunaga, S.; Shibasaki, M., J. Am. Chem. Soc. 2008, 130 (31), 10078-10079. (n) Sone, T.; Yamaguchi, A.; Matsunaga, S.; Shibasaki, M., Molecules 2012, 17 (2), 1617-1634.
3. (a) Sasai, H.; Kim, W. S.; Suzuki, T.; Shibasaki, M.; Mitsuda, M.; Hasegawa, J.; Ohashi, T., Tetrahedron Lett. 1994, 35 (33), 6123-6126. (b) Sasai, H.; Yamada, Y. M. A.; Suzuki, T.; Shibasaki, M., Tetrahedron 1994, 50 (43), 12313-12318. (c) Sasai, H.; Tokunaga, T.; Watanabe, S.; Suzuki, T.; Itoh, N.; Shibasaki, M., J. Org. Chem. 1995, 60 (23), 7388-7389. (d) Shimizu, S.; Ohori, K.; Arai, T.; Sasai, H.; Shibasaki, M., J. Org. Chem. 1998, 63 (21), 7547-7551.
4. (a) Aspinall, H. C.; Bickley, J. F.; Dwyer, J. L. M.; Greeves, N.; Kelly, R. V.; Steiner, A., Organometallics 2000, 19 (25), 5416-5423. (b) Wooten, A. J.; Salvi, L.; Carroll, P. J.; Walsh, P. J., Adv. Synth. Catal. 2007, 349 (4-5), 561-565.
5. (a) Di Bari, L.; Lelli, M.; Pintacuda, G.; Pescitelli, G.; Marchetti, F.; Salvadori, P., J. Am. Chem. Soc. 2003, 125 (18), 5549-5558. (b) Wooten, A. J.; Carroll, P. J.; Walsh, P. J., Angew. Chem. Int. Ed. 2006, 45 (16), 2549-2552. (c) Wooten, A. J.; Carroll, P. J.; Walsh, P. J., J. Am. Chem. Soc. 2008, 130 (23), 7407-7419.
6. Mashiko, T.; Kumagai, N.; Shibasaki, M., Org. Lett. 2008, 10 (13), 2725-2728.
7. (a) Davies, I. W.; Welch, C. J., Science 2009, 325 (5941), 701-704. (b) McNally, A.; Prier, C. K.; MacMillan, D. W. C., Science 2011, 334 (6059), 1114-1117. (c) Robbins, D. W.; Hartwig, J. F., Science 2011, 333 (6048), 1423-1427. (d) Schmink, J. R.; Bellomo, A.; Berritt, S., Aldrichimica Acta 2013, 46 (3), 71-80. (e) Zhang, J.; Stanciu, C.; Wang, B.; Hussain, M. M.; Da, C.-S.; Carroll, P. J.; Dreher, S. D.; Walsh, P. J., J. Am. Chem. Soc. 2011, 133 (50), 20552-20560. (f) McGrew, G. I.; Stanciu, C.; Zhang, J.; Carroll, P. J.; Dreher, S. D.; Walsh, P. J., Angew. Chem. Int. Edit. 2012, 51 (46), 11510-11513. (g) Zhang, J.; Bellomo, A.; Creamer, A. D.; Dreher, S. D.; Walsh, P. J., J. Am. Chem. Soc. 2012, 134 (33), 13765-13772. (h) Bellomo, A.; Zhang, J.; Trongsiriwat, N.; Walsh, P. J., Chem. Sci. 2013, 4 (2), 849-857. (i) Jia, T.; Bellomo, A.; Baina, K. E. L.; Dreher, S. D.; Walsh, P. J., J. Am. Chem. Soc. 2013, 135 (10), 3740-3743.
8. (a) Wooten, A. J.; Carroll, P. J.; Walsh, P. J., Org. Lett. 2007, 9 (17), 3359-3362. (b) Robinson, J. R.; Carroll, P. J.; Walsh, P. J.; Schelter, E. J., Angew. Chem. Int. Ed. 2012, 51 (40), 10159-10163. (c) Robinson, J. R.; Gordon, Z.; Booth, C. H.; Carroll, P. J.; Walsh, P. J.; Schelter, E. J., J. Am. Chem. Soc. 2013, 135 (50), 19016-19024. (d) Robinson, J. R.; Booth, C. H.; Carroll, P. J.; Walsh, P. J.; Schelter, E. J., Chem. Eur J. 2013, 19 (19), 5996-6004.
9. (a) Aakeroy, C. B.; Seddon, K. R., Chem. Soc. Rev. 1993, 22 (6), 397-407. (b) Steiner, T., Angew. Chem. Int. Ed. 2002, 41 (1), 48-76. (c) Meot-Ner, M., Chem. Rev. 2012, 112 (10), PR22-PR103.
10. (a) Bertini, I.; Gray, H. B.; Lippard, S. J.; Valentine, J. S., Biological Inorganic Chemistry: Structure and Reactivity. University Science Books: Sausalito, C A, 2007; Vol. 25. (b) Kabsch, W.; Sander, C., Biopolymers 1983, 22 (12), 2577-2637. (c) Schmidtchen, F. P.; Berger, M., Chem. Rev. 1997, 97 (5), 1609-1646. (d) MacBeth, C. E.; Golombek, A. P.; Young, V. G.; Yang, C.; Kuczera, K.; Hendrich, M. P.; Borovik, A. S., Science 2000, 289 (5481), 938-941. (e) Hirschberg, J. H. K. K.; Brunsveld, L.; Ramzi, A.; Vekemans, J. A. J. M.; Sijbesma, R. P.; Meijer, E. W., Nature 2000, 407 (6801), 167-170. (f) Moulton, B.; Zaworotko, M. J., Chem. Rev. 2001, 101 (6), 1629-1658. (g) Miller, B. G.; Wolfenden, R., Annu. Rev. Biochem. 2002, 71 (1), 847-885. (h) Schreiner, P. R., Chem. Soc. Rev. 2003, 32 (5), 289-296. (i) Taylor, M. S.; Jacobsen, E. N., Angew. Chem. Int. Ed. 2006, 45 (10), 1520-1543. (j) Lu, Y., Angew. Chem. Int. Ed. 2006, 45 (34), 5588-5601. (k) Natale, D.; Mareque-Rivas, J. C., Chem. Commun 2008, (4), 425-437. (1) Cordier, P.; Tournilhac, F.; Soulie-Ziakovic, C.; Leibler, L., Nature 2008, 451 (7181), 977-980. (m) So, Y.-M.; Wang, G.-C.; Li, Y.; Sung, H. H. Y.; Williams, I. D.; Lin, Z.; Leung, W.-H., Angew. Chem. Int. Ed. 2014, 53 (6), 1626-1629.
11. Margetic, D., Super Bases for Organic Synthesis: Guanidines, Amidines, Phosphazenes and Related Organocatalysts. Wiley: West Sussex, U K, 2009.
12. Bordwell, F. G., Acc. Chem. Res. 1988, 21 (12), 456-463.
13. (a) Hannon, C. L.; Anslyn, E. V., Bioorganic Chemistry Frontiers. Springer-Verlag: New York, 1993; Vol. 3. (b) Schug, K. A.; Lindner, W., Chem. Rev. 2004, 105 (1), 67-114.
14. Aspinall, H. C.; Dwyer, J. L. M.; Greeves, N.; Steiner, A., Organometallics 1999, 18 (8), 1366-1368.
15. Shannon, R. D., Acta Crystallogr., Sect. A: Found. Crystallogr. 1976, 32 (SEP1), 751-767.
16. (a) Cotton, F. A.; Day, V. W.; Hazen, E. E.; Larsen, S., J. Am. Chem. Soc. 1973, 95 (15), 4834-4840. (b) Cotton, F. A.; Day, V. W.; Hazen, E. E.; Larsen, S.; Wong, S. T. K., J. Am. Chem. Soc. 1974, 96 (14), 4471-4478. (c) Giacomelli, A.; Floriani, C.; Perego, G., Chem. Commun. 1982, (12), 650-652. (d) Janicki, R.; Starynowicz, P.; Mondry, A., Eur. J. Inorg. Chem. 2011, 2011 (24), 3601-3616. (e) Levin, J. R.; Gu, J.; Carroll, P. J.; Schelter, E. J., Dalton Trans. 2012, 41 (26), 7870-7872.
17. (a) Saa, J. M.; Tur, F.; Gonzalez, J.; Vega, M., Tetrahedron-Asymmetr 2006, 17 (1), 99-106. (b) Saa, J. M.; Tur, F.; Gonzalez, J., Chirality 2009, 21 (9), 836-842. (c) Di Bari, L.; Di Pietro, S.; Pescitelli, G.; Tur, F.; Mansilla, J.; Saa, J. M., Chem. Eur. J. 2010, 16 (47), 14190-14201.
18. (a) Richens, D. T., The Chemistry of Aqua Ions: Synthesis, Structure, and Reactivity. John Wiley & Sons: New York, 1997. (b) Aspinall, H. C., Chemistry of the f-block Elements. Overseas Publishing Company: UK, 2001; Vol. 1. (c) Cotton, S., Lanthanide and Actinide Chemistry. John Wiley & Sons Ltd: West Sussex, England, 2006.
19. (a) Baes, C. F.; Mesmer, R. E., The Hydrolysis of Cations. John Wiley & Sons: New York, 1976. (b) Rizkalla, E. N.; Choppin, G. R., Chapter 103 Hydration and hydrolysis of lanthanides. In Handbook on the Physics and Chemistry of Rare Earths, Karl A. Gschneidner, Jr.; LeRoy, E., Eds. Elsevier: 1991; Vol. Volume 15, pp 393-442. (c) Rizkalla, E. N.; Choppin, G. R., Chapter 127 Lanthanides and actinides hydration and hydrolysis. In Handbook on the Physics and Chemistry of Rare Earths, Karl A. Gschneidner, J. L. E. G. R. C.; Lander, G. H., Eds. Elsevier: 1994; Vol. Volume 18, pp 529-558.
20. (a) Caulton, K. G.; Hubert-Pfalzgraf, L. G., Chem. Rev. 1990, 90 (6), 969-995. (b) Mehrotra, R. C.; Singh, A.; Tripathi, U. M., Chem. Rev. 1991, 91 (6), 1287-1303. (c) Evans, W. J.; Sollberger, M. S.; Ziller, J. W., J. Am. Chem. Soc. 1993, 115 (10), 4120-4127. (d) Schumann, H.; Meese-Marktscheffel, J. A.; Esser, L., Chem. Rev. 1995, 95 (4), 865-986. (e) Bünzli, J.-C. G.; Piguet, C., Chem. Rev. 2002, 102 (6), 1897-1928. (f) Kempe, R.; Noss, H.; Irrgang, T., J. Organomet. Chem. 2002, 647 (1-2), 12-20. (g) Arnold, P. L.; Casely, I. J., Chem. Rev. 2009, 109 (8), 3599-3611. (h) Shibasaki, M.; Kanai, M.; Matsunaga, S.; Kumagai, N., Acc. Chem. Res. 2009, 42 (8), 1117-1127. (i) Mandal, S. K.; Roesky, H. W., Acc. Chem. Res. 2010, 43 (2), 248-259. (j) Zimmermann, M.; Anwander, R., Chem. Rev. 2010, 110 (10), 6194-6259. (k) Oelkers, B.; Butovskii, M. V.; Kempe, R., Chem. Eur J. 2012, 18 (43), 13566-13579. (1) Arnold, P. L.; Hollis, E.; Nichol, G. S.; Love, J. B.; Griveau, J.-C.; Caciuffo, R.; Magnani, N.; Maron, L.; Castro, L.; Yahia, A.; Odoh, S. O.; Schreckenbach, G., J. Am. Chem. Soc. 2013, 135 (10), 3841-3854.
21. (a) Jung, M. E., 1.1-Stabilized Nucleophiles with Electron Deficient Alkenes and Alkynes. In Comprehensive Organic Synthesis, Trost, B. M.; Fleming, I., Eds. Pergamon: Oxford, 1991; pp 1-67. (b) Krause, N.; Hoffmann-Roder, A., Synthesis 2001, (2), 171-196. (c) Kanai, M.; Shibasaki, M., Asymmetric Carbon-Carbon Bond-Forming Reactions: Asymmetric Michael Reactions. In Catalytic Asymmetric Synthesis, John Wiley & Sons, Inc.: 2005; pp 569-592. (d) Howell, G. P., Org. Process Res. Dev. 2012, 16 (7), 1258-1272.
22. Sasai, H.; Emori, E.; Arai, T.; Shibasaki, M., Tetrahedron Lett. 1996, 37 (31), 5561-5564.
23. (a) Tzvetkov, N. T.; Schmoldt, P.; Neumann, B.; Stammler, H. G.; Mattay, J., Tetrahedron-Asymmetr 2006, 17 (6), 993-998. (b) De Buysser, F.; Verlinden, L.; Verstuyf, A.; De Clercq, P. J., Tetrahedron Lett. 2009, 50 (28), 4174-4177. (c) Abele, S.; Inauen, R.; Funel, J. A.; Weller, T., Org. Process Res. Dev. 2012, 16 (1), 129-140.
24. (a) Ohori, K.; Shimizu, S.; Ohshima, T.; Shibasaki, M., Chirality 2000, 12 (5-6), 400-403. (b) Ohshima, T.; Xu, J. Y.; Takita, R.; Shimizu, S.; Zhong, D. F.; Shibasaki, M., J. Am. Chem. Soc. 2002, 124 (49), 14546-14547.
25. Jiricek, J.; Blechert, S., J. Am. Chem. Soc. 2004, 126(11), 3534-3538.
26. Matveenko, M.; Liang, G. X.; Lauterwasser, E. M. W.; Zubia, E.; Trauner, D., J. Am. Chem. Soc. 2012, 134 (22), 9291-9295.
27. Haruta, Y.; Onizuka, K.; Watanabe, K.; Kono, K.; Nohara, A.; Kubota, K.; Imoto, S.; Sasaki, S., Tetrahedron 2008, 64 (30-31), 7211-7218.
28. Walsh, P. J.; Li, H. M.; de Parrodi, C. A., Chem. Rev. 2007, 107 (6), 2503-2545.
29. (a) Arai, T.; Sasai, H.; Yamaguchi, K.; Shibasaki, M., J. Am. Chem. Soc. 1998, 120 (2), 441-442. (b) Xu, Y.; Ohori, K.; Ohshima, T.; Shibasaki, M., Tetrahedron 2002, 58 (13), 2585-2588.
30. Tsogoeva, S. B., Eur J. Org. Chem. 2007, 2007 (11), 1701-1716.
31. (a) Hamashima, Y.; Hotta, D.; Sodeoka, M., J. Am. Chem. Soc. 2002, 124 (38), 11240-11241. (b) Ogawa, C.; Kizu, K.; Shimizu, H.; Takeuchi, M.; Kobayashi, S., Chem. Asian J. 2006, 1 (1-2), 121-124. (c) Akiyama, T.; Katoh, T.; Mori, K., Angew. Chem. Int. Ed. 2009, 48 (23), 4226-4228. (d) Yang, J. J.; Li, W. J.; Jin, Z. C.; Liang, X. M.; Ye, J. X., Org. Lett. 2010, 12 (22), 5218-5221.
32. (a) Mynderse, J. S.; Hunt, A. H.; Moore, R. E., J. Nat. Prod 1988, 51 (6), 1299-1301. (b) Pettit, G. R.; Kamano, Y.; Kizu, H.; Dufresne, C.; Herald, C. L.; Bontems, R. J.; Schmidt, J. M.; Boettner, F. E.; Nieman, R. A., Heterocycles 1989, 28 (2), 553-558. (c) Hart, D. J.; Ha, D. C., Chem. Rev. 1989, 89 (7), 1447-1465. (d) Cardillo, G.; Tomasini, C., Chem. Soc. Rev. 1996, 25 (2), 117-128. (e) Benedetti, F.; Norbedo, S., Chem. Commun. 2001, (2), 203-204. (f) Luesch, H.; Williams, P. G.; Yoshida, W. Y.; Moore, R. E.; Paul, V. J., J. Nat. Prod 2002, 65 (7), 996-1000. (g) Zaborenko, N.; Bedore, M. W.; Jamison, T. F.; Jensen, K. F., Org. Process Res. Dev. 2010, 15 (1), 131-139.
33. Yamagiwa, N.; Matsunaga, S.; Shibasaki, M., J. Am. Chem. Soc. 2003, 125 (52), 16178-16179.
34. (a) Arai, T.; Yamada, Y. M. A.; Yamamoto, N.; Sasai, H.; Shibasaki, M., Chem. Eur. J. 1996, 2 (11), 1368-1372. (b) Vogl, E. M.; Gröger, H.; Shibasaki, M., Angew. Chem. Int. Ed. 1999, 38 (11), 1570-1577. (c) Tian, J.; Yamagiwa, N.; Matsunaga, S.; Shibasaki, M., Angew. Chem. Int. Ed. 2002, 41 (19), 3636-3638. (d) Sone, T.; Lu, G.; Matsunaga, S.; Shibasaki, M., Angew. Chem. Int. Ed. 2009, 48 (9), 1677-1680.
35. As of 02/17/14, the price per gram of La (www.strem.com) was $0.90 for La(NO₃)₃.6H₂O (100 g quantity), as compared to $150.23 for La(OⁱPr)₃ (1 g quantity) or $263.37 for La[N(SiMe₃)₂]₃ (1 g quantity). Please see Table SI for a more detailed comparison Supporting information
36. (a) Tomioka, K.; Seo, W.; Ando, K.; Koga, K., Tetrahedron Lett. 1987, 28 (52), 6637-6640. (b) Kim, Y. S.; Matsunaga, S.; Das, J.; Sekine, A.; Ohshima, T.; Shibasaki, M., J. Am. Chem. Soc. 2000, 122 (27), 6506-6507.
37. Ton, T. M. U.; Tejo, C.; Tiong, D. L. Y.; Chan, P. W. H., J. Am. Chem. Soc. 2012, 134 (17), 7344-7350.
38. Nepali, K.; Kadian, K.; Ojha, R.; Dhiman, R.; Garg, A.; Singh, G.; Buddhiraja, A.; Bedi, P. M. S.; Dhar, K. L., Med. Chem. Res. 2012, 21, 2990-2997.
39. Bradley, D. C.; Ghotra, J. S.; Hart, F. A., Dalton Trans. 1973, (10), 1021-1027.
40. Brüker, SAINT. Brüker AXS Inc.: Madison, Wis., USA, 2009.
41. Brüker, SHELXTL. Brüker AXS Inc.: Madison, Wis., USA, 2009.
42. Sheldrick, G. M., TWINABS. University of Gottingen, Germany, 2008.
43. Sheldrick, G. M., SADABS. University of Gottingen, Germany, 2007.
44. Sheldrick, G. M., Acta Crystallogr. 2008, 64, 112-122.
45. Sluis, V. D. P.; Spek, A. L., Acta Crystallographica 1990, A46, 194.
46. Kreidler, B.; Baro, A.; Christoffers, J., Eur. J. Org. Chem. 2005, 2005 (24), 5339-5348.
47. Cheon, C. H.; Yamamoto, H., Tetrahedron 2010, 66 (24), 4257-4264.
48. (a) Aspinall, H. C., Chemistry of the f-block Elements; Overseas Publishing Co.: UK, 2001; Vol. 1; (b) Cotton, S., Lanthanide and Actinide Chemistry; John Wiley & Sons Ltd: West Sussex, England, 2006.

1. A precatalyst complex of the formula: 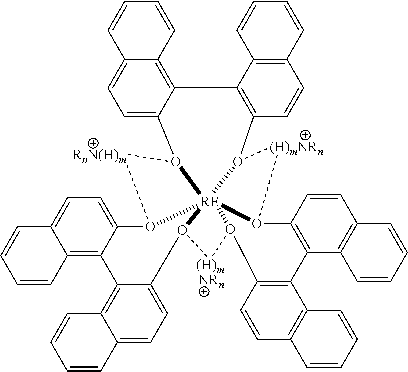
wherein RE represents a rare earth element, NRₙ represents an amine base, m=1 or 2, n=1, 2 or 3 and m+n≦4; and the dashed lines indicate hydrogen bonding which may be monodentate or bidentate hydrogen bonding.

2. The precatalyst of claim 1, wherein RE is a rare earth element selected from Sc, Y and La through Lu.

3. The precatalyst of claim 1, wherein the amine base is selected from the group of a guanidine, an amidine and a heterocyclic amine, said amidine being cyclic or non-cyclic.

4. A process for preparing the precatalyst complex of claim 1 by forming a reaction mixture comprising (i) a rare earth-containing reactant selected from the group of RE(NO₃)₃, RE(N[SiMe₃]₂); (ii) (S)1,1′-bi-2-naphthol((S)-BINOL) and (iii) NR₃ in a suitable solvent, and subjecting said mixture to conditions yielding said complex.

5. A process for generating a REMB catalyst comprising reacting the precatalyst complex of claim 1 with a metal halide or a pseudo-halide to yield said REMB catalyst.

6. The process of claim 5, wherein said metal halide is an alkali metal halide.

7. The process of claim 5, wherein said pseudo-halide comprises an alkali metal cation and an anion selected from the group of tetrafluoroborate, hexafluorophosphate, tetrakis-(3,5-bis(trifluoromethyl)) borate.

8. A process for generating a REMB catalyst comprising reacting the precatalyst complex of claim 1 with M(N[SiMe₃]₂), M being an alkali metal, to yield said REMB catalyst.

9. The catalyst produced by the process of claim 5.

10. In a chemical reaction comprising the base-promoted conjugate addition of a carbon nucleophile/donor to an activated, unsaturated compound/acceptor, the improvement which comprises catalyzing said reaction using the catalyst of claim 9.

11. In a chemical reaction comprising the 1,4-addition of a dienophile double bond to a conjugated dien to yield a 6-membered ring compound having at least one chiral center, the improvement which comprises catalyzing said reaction using the catalyst of claim 9.

12. A catalyzed chemical reaction selected from the group of a Michael addition reaction, an aza-Michael addition reaction and a direct Aldol reaction, wherein the catalyst in the reaction is generated from the precatalyst of claim 1.
